(19) United States
(12) Patent Application Publication
(10) Pub. No.: US 2003/0055072 A1
(43) Pub. Date: Mar. 20, 2003

(54) Methods of inhibiting Pin1-associated states using a fredericamycin a compound

(76) Inventors: Kun Ping Lu, Newton, MA (US); Gunter Fischer, Halle (DE)

Correspondence Address: LAHIVE & COCKFIELD, 28 STATE STREET, BOSTON, MA, 02109, US

(21) Appl. No.: 10/027,864

(22) Filed: Dec. 21, 2001

Related U.S. Application Data
(60) Provisional application No. 60/342,572, filed on Dec. 20, 2001.
(60) Provisional application No. 60/257,412, filed on Dec. 22, 2000.

(51) Int. Cl. A61K 31/4747

(52) U.S. Cl. 514/278

(57) ABSTRACT

This invention provides a method for treating a Pin1-associated state in a subject including administering to a subject an effective amount of a fredericamycin A compound such that the Pin1-associated state is treated. In another aspect, this invention includes the above described method, wherein the Pin1-associated state is a cyclin D1 elevated state, neoplastic transformation, and/or tumor growth. In an embodiment, this invention provides the above described methods, wherein the Pin1-associated state is colon cancer, breast cancer, a sarcoma, a malignant lymphoma, and/or esophageal cancer. This invention also provides a method for treating cyclin D1 overexpression in a subject including administering to a subject an effective amount of a combination of a fredericamycin A compound and a hyperplastic inhibitory agent such that the cyclin D1 overexpression is treated.

### RELATED APPLICATIONS

This application claims priority to U.S. Provisional Patent Application No. ______, filed on Dec. 20, 2001, entitled “Methods of Inhibiting Pin1-Associated States Using a Fredericamycin A Compound;” and U.S. Provisional Patent Application No. 60/257,412, filed on Dec. 22, 2000. This application is related to U.S. patent application Ser. No. 09/726,464, filed Nov. 29, 2000; U.S. application Ser. No. 08/988,842, filed Dec. 11, 1997; and WO 99/12962, published Mar. 8, 1999. The entire contents of each of the aforementioned applications are hereby incorporated herein by reference.

### BACKGROUND OF THE INVENTION

At the center of the cell cycle are the cyclin dependent kinases (cdks). The cdks are a family of structurally related small protein (˜34-40 kD) kinase catalytic subunits whose activation requires association with a cyclin regulatory subunit. In most cases, full activation also requires phosphorylation of a threonine near the kinase active site. Cdk function has been well conserved during evolution. For example, yeast cells can divide normally when their cdk1 gene is replaced with the human cdk1 gene. The cdks form unique complexes with cyclins and those complex promote cell proliferation by phosphorylating specific substrates in a cell cycle dependent fashion to ensure progression through various cell cycle transitions. The precise timing of cyclin-cdk activity during the cell cycle determines whether the cell cycle continues or becomes blocked. Morgan 1997. Annu. Rev. Cell. Dev. Biol 13:261-291.

There are at least 11 different mammalian cyclins including cyclins A, B1, B2, C, D1, D2, D3, E, F, G, and H. The different cyclins reach peak activity during different phases of the cell cycle. Cyclin D1 is a protein derived from the PRAD1, CCND1, or bc1-1 gene on chromosome 11q13. The cyclin D1 gene spans about 15 kb and has 5 exons. Its upstream region has Sp1 binding sites, a potential E2F binding motif, and no obvious TATA box. Cyclin D1 reaches its maximum activity during mid G₁ phase, decreases during S-phase, and remains low throughout the rest of the cycle. Cyclin D1 appears to regulate the transition from the G₁ to S phase of the cell cycle. Donnellan, et al. 1998. J. Clin. Pathol: Mol. Pathol. 51:1-7. In normal cells, the level of cyclin D1 protein fluctuates in response to external stimuli. In contrast, expression is unscheduled in transformed cell lines and may occur throughout the cycle.

Increased cyclin D1 expression has been found in a vast range of primary human tumors. Increased cyclin D1 expression has been detected in the form of gene amplification, increased cyclin D1 RNA expression, and increased cyclin D1 protein expression. Most clinical studies comparing cyclin D1 gene amplification with expression of cyclin D1 have found that more cases show over-expression of both RNA and protein than show amplification of the gene. The presence of increased cyclin D1RNA and/or protein expression without gene amplification suggests that other cellular genes such as pRb may affect the expression cyclin D1. Human tumors found to have increased cyclin D1 expression include: parathyroid adenomas, mantle cell lymphomas, breast cancers, head and neck squamous cell carcinomas (i.e. squamous carcinomas in the oral cavity, nasopharynx, pharynx, hypopharynx, and larynx), esophageal cancers, hepatocellular carcinomas, colorectal cancers, genitourinary cancers, lung cancers (i.e. squamous cell carcinomas of the lung), skins cancers (i.e. squamous cell carcinomas, melanomas, and malignant fibrous histiocytomas), sarcomas, and central nervous system malignancies (i.e. astrocytomas and glioblastomas), gastric adenocarcinomas, pancreatic adenocarcinomas, squamous carcinomas of the gall bladder. Donnellan, et al. 1998. J. Clin. Pathol: Mol. Pathol. 51:1-7. The cyclin D1 gene is amplified in approximately 20% of mammary carcinomas and the protein is overexpressed in approximately 50% of mammary carcinomas. Barnes, et al. 1998. Breast Cancer Research and Treatment. 52: 1-15. It is believed that in many tumors, cyclin D1 acts in co-operation with other oncogenes or tumor suppressor genes.

### SUMMARY OF THE INVENTION

This invention provides a method for treating a Pin1-associated state in a subject including administering to a subject an effective amount of a fredericamycin A compound such that the Pin1-associated state is treated.

In another aspect, this invention includes the above described method, wherein the Pin1-associated state is a cyclin D1 elevated state, neoplastic transformation, and/or tumor growth.

This invention also encompasses the above described methods, wherein the treating includes inhibiting tumor growth, preventing the occurrence of tumor growth in the subject, or reducing the growth of a pre-existing tumor in the subject. In an embodiment, this invention provides the above described methods, wherein the Pin1-associated state is cancer, e.g., colon cancer, breast cancer, a sarcoma, a malignant lymphoma, and/or esophageal cancer.

This invention also encompasses the above described methods, wherein the Pin1-associated state is caused by overexpression of Pin1, DNA damage, an oncogenic protein, and/or Ha-Ras.

This invention further includes a method for treating cyclin D1 overexpression in a subject including administering to a subject an effective amount of a fredericamycin A compound such that cyclin D1 overexpression is treated.

This invention also features the above described methods, wherein the cyclin D1 overexpression results in neoplastic transformation and/or tumor growth.

This invention provides the above described methods, wherein the treating includes inhibiting tumor growth, preventing the occurrence of tumor growth in the subject, and/or reducing the growth of a pre-existing tumor in the subject.

This invention further encompasses the above described methods, wherein the cyclin D1 overexpression results in colon cancer, breast cancer, sarcoma, malignant lymphoma, and/or esophageal cancer.

This invention also includes the above described methods, wherein the cyclin D1 overexpression is caused by overexpression of Pin1, DNA damage, an oncogenic protein, and/or Ha-Ras.

In another aspect, this invention also encompasses a method for treating tumor growth in a subject including administering to a subject an effective amount of a fredericamycin A compound having Formula VI

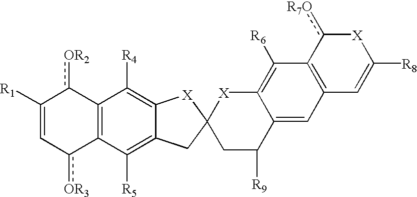

wherein the dotted lines indicate optional double bonds;

X is N, O, S, or C;

R₁, R₄, R₅, R₆, R₈, and R₉ are independently hydrogen, alkyl, hydroxyl, alkoxy, alkanoyl, alkoxycarbonyl, alkylcarbonyl, alkylcarbonyloxy, alkoxycarbonyloxy; and

R₂, R₃, and R₇ are independently hydrogen, alkyl, alkanoyl, or nothing; such that the tumor growth is treated.

In an embodiment, this invention also includes a packaged Pin1-associated state treatment, including a fredericamycin A compound packaged with instructions for using an effective amount of the fredericamycin A compound to treat a Pin1-associated state.

This invention further encompasses a packaged cyclin D1 overexpression treatment, including a fredericamycin A compound packaged with instructions for using an effective amount of the fredericamycin A compound to treat cyclin D1 overexpression.

This invention also features a packaged cancer treatment, including a fredericamycin A compound packaged with instructions for using an effective amount of the fredericamycin A compound to treat cancer.

In an embodiment, this invention provides a method for treating a Pin1-associated state in a subject including administering to a subject an effective amount of a combination of a fredericamycin A compound and a hyperplastic inhibitory agent such that the Pin1-associated state is treated.

In an embodiment, this invention encompasses the above described methods, wherein the hyperplastic inhibitory agent is tamoxifen, paclitaxel, docetaxel, interleukin-2, rituximab, tretinoin, and/or methotrexate.

In another aspect, this invention further includes a method for treating cancer in a subject including administering to a subject an effective amount of a combination of a fredericamycin A compound and a hyperplastic inhibitory agent such that the cancer is treated.

This invention also provides a method for treating cyclin D1 overexpression in a subject including administering to a subject an effective amount of a combination of a fredericamycin A compound and a hyperplastic inhibitory agent such that the cyclin D1 overexpression is treated.

This invention also features the above described methods, wherein the fredericamycin A compound has Formula IX

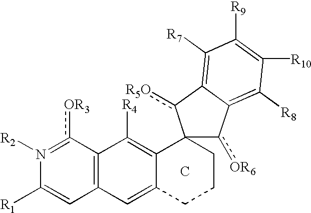

wherein the dotted lines around C indicate that C may be a 5 or 6 membered ring;

wherein the dotted lines not around C indicate optional double bonds;

R₁ is alkyl, alkenyl, alkanoyl, alknyl;

R₂ is hydrogen or alkyl;

R₉ and R₁₀ are both hydrogen or together form a ring having the structure

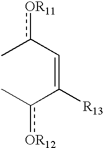

R₃, R₅, R₆, R₁₁, and R₁₂ are independently hydrogen, alkyl, alkanoyl, or nothing; and R₄, R₇, R₈, R₁₃ are independently hydrogen, alkyl, hydroxyl, alkoxy, alkanoyl, alkoxycarbonyl, alkylcarbonyl, alkylcarbonyloxy, alkoxycarbonyloxy. This invention provides the above described methods, wherein the fredericamycin A compound is fredericamycin A.

### BRIEF DESCRIPTION OF THE DRAWINGS

FIG. 1 shows a plot of hPin1 activity (%) versus fredericamycin A concentration (μM) as described in the example below.

FIG. 2 shows a plot of hPin1 activity (BE) versus time (min) as described in the example below.

FIG. 3 shows a graph of the hPin1 activity (%) of 209 nM of hPin1 incubated with 0 (▪) and 0.16 (□) mM fredericamycin A with the PPIase activity of hPin1 measured before and after micro-separation through a semi-permeable membrane as described in the example below.

FIG. 4 is a line graph of mean tumor volume (cm³) showing the effect of Fredricamycin on DU-145 prostate tumor bearing scid mice.

FIG. 5 is a line graph of mean mouse weight (g) showing the effect of Fredricamycin on DU-145 prostate tumor bearing scid mice.

### DETAILED DESCRIPTION OF THE INVENTION

Chemistry Terminology

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone. In an embodiment, a straight chain or branched chain alkyl has 10 or fewer carbon atoms in its backbone (e.g., C₁-C₁₀ for straight chain, C₃-C₁₀ for branched chain), and more preferably 6 or fewer. Likewise, preferred cycloalkyls have from 4-7 carbon atoms in their ring structure, and more preferably have 5 or 6 carbons in the ring structure.

Moreover, the term alkyl includes both “unsubstituted alkyls” and “substituted alkyls”, the latter of which refers to alkyl moieties having substituents replacing a hydrogen on one or more carbons of the hydrocarbon backbone. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “aralkyl” moiety is an alkyl substituted with an aryl (e.g., phenylmethyl (benzyl)). The term “alkyl” also includes the side chains of natural and unnatural amino acids. Examples of halogenated alkyl groups include fluoromethyl, difluoromethyl, trifluoromethyl, chloromethyl, dichloromethyl, trichloromethyl, perfluoromethyl, perchloromethyl, perfluoroethyl, perchloroethyl, etc.

The term “aryl” includes groups, including 5- and 6-membered single-ring aromatic groups that may include from zero to four heteroatoms, for example, benzene, phenyl, pyrrole, furan, thiophene, thiazole, isothiaozole, imidazole, triazole, tetrazole, pyrazole, oxazole, isooxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, napthridine, indole, benzofuran, purine, benzofuran, deazapurine, or indolizine. Those aryl groups having heteroatoms in the ring structure may also be referred to as “aryl heterocycles”, “heterocycles,” “heteroaryls” or “heteroaromatics”. The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkylaminoacarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The term “alkenyl” includes unsaturated aliphatic groups analogous in length and possible substitution to the alkyls described above, but that contain at least one double bond.

For example, the term “alkenyl” includes straight-chain alkenyl groups (e.g., ethenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term alkenyl further includes alkenyl groups which include oxygen, nitrogen, sulfur or phosphorous atoms replacing one or more carbons of the hydrocarbon backbone. In certain embodiments, a straight chain or branched chain alkenyl group has 6 or fewer carbon atoms in its backbone (e.g., C₂-C₆ for straight chain, C₃-C₆ for branched chain). Likewise, cycloalkenyl groups may have from 3-8 carbon atoms in their ring structure, and more preferably have 5 or 6 carbons in the ring structure. The term C₂-C₆ includes alkenyl groups containing 2 to 6 carbon atoms.

Moreover, the term alkenyl includes both “unsubstituted alkenyls” and “substituted alkenyls”, the latter of which refers to alkenyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond.

For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. The term alkynyl further includes alkynyl groups which include oxygen, nitrogen, sulfur or phosphorous atoms replacing one or more carbons of the hydrocarbon backbone. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

Moreover, the term alkynyl includes both “unsubstituted alkynyls” and “substituted alkynyls”, the latter of which refers to alkynyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Unless the number of carbons is otherwise specified, “lower alkyl” as used herein means an alkyl group, as defined above, but having from one to five carbon atoms in its backbone structure. “Lower alkenyl” and “lower alkynyl” have chain lengths of, for example, 2-5 carbon atoms.

The term “acyl” includes compounds and moieties which contain the acyl radical (CH₃CO—) or a carbonyl group. The term “substituted acyl” includes acyl groups where one or more of the hydrogen atoms are replaced by for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “acylamino” includes moieties wherein an acyl moiety is bonded to an amino group. For example, the term includes alkylcarbonylamino, arylcarbonylamino, carbamoyl and ureido groups.

The term “aroyl” includes compounds and moieties with an aryl or heteroaromatic moiety bound to a carbonyl group. Examples of aroyl groups include phenylcarboxy, naphthyl carboxy, etc.

The terms “alkoxyalkyl”, “alkylaminoalkyl” and “thioalkoxyalkyl” include alkyl groups, as described above, which further include oxygen, nitrogen or sulfur atoms replacing one or more carbons of the hydrocarbon backbone, e.g., oxygen, nitrogen or sulfur atoms.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups and may include cyclic groups such as cyclopentoxy. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc.

The term “amine” or “amino” includes compounds where a nitrogen atom is covalently bonded to at least one carbon or heteroatom. The term “alkyl amino” includes groups and compounds wherein the nitrogen is bound to at least one additional alkyl group. The term “dialkyl amino” includes groups wherein the nitrogen atom is bound to at least two additional alkyl groups. The term “arylamino” and “diarylamino” include groups wherein the nitrogen is bound to at least one or two aryl groups, respectively. The term “alkylarylamino,” “alkylaminoaryl” or “arylaminoalkyl” refers to an amino group which is bound to at least one alkyl group and at least one aryl group. The term “alkaminoalkyl” refers to an alkyl, alkenyl, or alkynyl group bound to a nitrogen atom which is also bound to an alkyl group.

The term “amide” or “aminocarboxy” includes compounds or moieties which contain a nitrogen atom which is bound to the carbon of a carbonyl or a thiocarbonyl group. The term includes “alkaminocarboxy” groups which include alkyl, alkenyl, or alkynyl groups bound to an amino group bound to a carboxy group. It includes arylaminocarboxy groups which include aryl or heteroaryl moieties bound to an amino group which is bound to the carbon of a carbonyl or thiocarbonyl group. The terms “alkylaminocarboxy,” “alkenylaminocarboxy,” “alkynylaminocarboxy,” and “arylaminocarboxy” include moieties wherein alkyl, alkenyl, alkynyl and aryl moieties, respectively, are bound to a nitrogen atom which is in turn bound to the carbon of a carbonyl group.

The term “carbonyl” or “carboxy” includes compounds and moieties which contain a carbon connected with a double bond to an oxygen atom, and tautomeric forms thereof. Examples of moieties which contain a carbonyl include aldehydes, ketones, carboxylic acids, amides, esters, anhydrides, etc. The term “carboxy moiety” or “carbonyl moiety” refers to groups such as “alkylcarbonyl” groups wherein an alkyl group is covalently bound to a carbonyl group, “alkenylcarbonyl” groups wherein an alkenyl group is covalently bound to a carbonyl group, “alkynylcarbonyl” groups wherein an alkynyl group is covalently bound to a carbonyl group, “arylcarbonyl” groups wherein an aryl group is covalently attached to the carbonyl group. Furthermore, the term also refers to groups wherein one or more heteroatoms are covalently bonded to the carbonyl moiety. For example, the term includes moieties such as, for example, aminocarbonyl moieties, (wherein a nitrogen atom is bound to the carbon of the carbonyl group, e.g., an amide), aminocarbonyloxy moieties, wherein an oxygen and a nitrogen atom are both bond to the carbon of the carbonyl group (e.g., also referred to as a “carbamate”). Furthermore, aminocarbonylamino groups (e.g., ureas) are also include as well as other combinations of carbonyl groups bound to heteroatoms (e.g., nitrogen, oxygen, sulfur, etc. as well as carbon atoms). Furthermore, the heteroatom can be further substituted with one or more alkyl, alkenyl, alkynyl, aryl, aralkyl, acyl, etc. moieties.

The term “thiocarbonyl” or “thiocarboxy” includes compounds and moieties which contain a carbon connected with a double bond to a sulfur atom. The term “thiocarbonyl moiety” includes moieties which are analogous to carbonyl moieties. For example, “thiocarbonyl” moieties include aminothiocarbonyl, wherein an amino group is bound to the carbon atom of the thiocarbonyl group, furthermore other thiocarbonyl moieties include, oxythiocarbonyls (oxygen bound to the carbon atom), aminothiocarbonylamino groups, etc.

The term “ether” includes compounds or moieties which contain an oxygen bonded to two different carbon atoms or heteroatoms. For example, the term includes “alkoxyalkyl” which refers to an alkyl, alkenyl, or alkynyl group covalently bonded to an oxygen atom which is covalently bonded to another alkyl group.

The term “ester” includes compounds and moieties which contain a carbon or a heteroatom bound to an oxygen atom which is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc. The alkyl, alkenyl, or alkynyl groups are as defined above.

The term “thioether” includes compounds and moieties which contain a sulfur atom bonded to two different carbon or hetero atoms. Examples of thioethers include, but are not limited to alkthioalkyls, alkthioalkenyls, and alkthioalkynyls. The term “alkthioalkyls” include compounds with an alkyl, alkenyl, or alkynyl group bonded to a sulfur atom which is bonded to an alkyl group. Similarly, the term “alkthioalkenyls” and alkthioalkynyls” refer to compounds or moieties wherein an alkyl, alkenyl, or alkynyl group is bonded to a sulfur atom which is covalently bonded to an alkynyl group.

The term “hydroxy” or “hydroxyl” includes groups with an —OH or —O⁻.

The term “halogen” includes fluorine, bromine, chlorine, iodine, etc. The term “perhalogenated” generally refers to a moiety wherein all hydrogens are replaced by halogen atoms.

The terms “polycyclyl” or “polycyclic radical” include moieties with two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkylaminoacarbonyl, aralkylaminocarbonyl, alkenylaminocarbonyl, alkylcarbonyl, arylcarbonyl, aralkylcarbonyl, alkenylcarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkyl, alkylaryl, or an aromatic or heteroaromatic moiety.

The term “heteroatom” includes atoms of any element other than carbon or hydrogen. Preferred heteroatoms are nitrogen, oxygen, sulfur and phosphorus.

The term “heterocycle” or “heterocyclic” includes saturated, unsaturated, aromatic (“heteroaryls” or “heteroaromatic”) and polycyclic rings which contain one or more heteroatoms. Examples of heterocycles include, for example, benzodioxazole, benzofuran, benzoimidazole, benzothiazole, benzothiophene, benzoxazole, deazapurine, furan, indole, indolizine, imidazole, isooxazole, isoquinoline, isothiaozole, methylenedioxyphenyl, napthridine, oxazole, purine, pyrazine, pyrazole, pyridazine, pyridine, pyrimidine, pyrrole, quinoline, tetrazole, thiazole, thiophene, and triazole. Other heterocycles include morpholine, piprazine, piperidine, thiomorpholine, and thioazolidine. The heterocycles may be substituted or unsubstituted. Examples of substituents include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkylaminoacarbonyl, aralkylaminocarbonyl, alkenylaminocarbonyl, alkylcarbonyl, arylcarbonyl, aralkylcarbonyl, alkenylcarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkyl, alkylaryl, or an aromatic or heteroaromatic moiety.

It will be noted that the structure of some of the compounds of this invention includes asymmetric carbon atoms. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis. Furthermore, the structures and other compounds and moieties discussed in this application also include all tautomers thereof.

Fredericamycin A Compounds

“Fredericamycin A compound” is intended to include fredericamycin A and compounds which are structurally similar to fredericamycin A and/or analogs of fredericamycin A. The language “fredericamycin A compound” can also include “mimics” or “inhibitors of fredericamycin A.” “Mimics” is intended to include compounds which may not be structurally similar to fredericamycin A but mimic the therapeutic activity of fredericamycin A or structurally similar fredericamycin A compounds in vivo. The “inhibitors of fredericamycin A” are compounds which inhibit the activity of fredericamycin A. The fredericamycin A compounds of this invention are those compounds which are useful for inhibiting Pin1 in subjects (patients). The term fredericamycin A compound also is intended to include pharmaceutically acceptable salts of the compounds. Fredericamycin A compounds can be naturally occurring or chemically synthesized.

Fredericamycin A can be isolated from a strain of Streptomyces griseus. In one procedure for the isolation of crude fredericamycin A, a whole broth from various fermentation runs is centrifuged to separate the mycelium from the broth. The pH of the filtered broth is adjusted to 2.0 with dilute sulfuric acid. It is left at 4° C. for 96 hours and the precipitated fredericamycin A is filtered off. The filtrate is then extracted with ethyl acetate 2 times. The mycelium is suspended in water and homogenized in a blender. The pH of the mixture is adjusted to 2.0 with dilute sulfuric acid and extracted with ethyl acetate. The mixture is filtered, the ethyl acetate extract is separated, and the aqueous phase is discarded. The extraction and purification of fredericamycin A from Streptomyces griseus is described in detail in Pandey, et. al. 1981. J Antibiot. 34(11):1389-401.

Numerous references describe the synthesis of fredericamycin A including: Kita, et al. 1998. J Synth. Organic Chem. Jpn. 56:963-974; Boger. 1996. J Heterocyclic Chem. 33:1519-1531; Boger, et al. 1995. J. Am. Chem. Soc. 117:11839-11849; Clive, et al. 1994. J. Am. Chem. Soc. 116:11275-11286; Wendt, et al. 1994. J. Am. Chem. Soc. 116:9921-9926; Rao, et al. 1994. Heterocycles. 37:1893-1912; Saintjalmes, et al. 1993. Bulletin de la Societe Chimique De France. 130:447-449; Clive, et al. Oct. 15, 1992. J. Chem. Soc. Chem. Comms. N20 pp. 1489-1490; Wendt., et al. 1994. J. Am. Chem. Soc. 116:9921-6; Kelly, et al. 1988. J. Am. Chem. Soc. 110:6471-80; Rama, et al. 1994. Heterocycles 37:1893-1912; Kelly, et al. 1988. J. Am. Chem. Soc. 110:6471-6480; Rama, et al. 1984. J. Chem. Soc. Chem. Comms. N16 pp. 1119-1120; Clive, et al. 1995. Stud. Nat. Prod. Chem. 16:27-74; and Kelly, et al. 1986. J. Am. Chem. Soc. 108:7100-7101.

“Fredericamycin A compounds” which are derivatives of fredericamycin A and their synthesis are described in Yokoi, et al, U.S. Pat. No. 4,584,377; Kelly, et al, U.S. Pat. No. 5,166,208; Clive, et al. 1996. Tetrahedron 52:6085-6116; Evans, et al. 1988. J. Org. Chem. 53:5519-27; Clive, et al. 1987. J. Org. Chem. 52:1339-1342; Clive, et al. 1987. J. Heterocylic. Chem. 24:509-511; Bennett, et al. 1986. J. Chem. Soc. Chem. Comms. N11 pp. 878-880; Braun, et al. 1986. Tetrahedron Letters 27:179-182; Kita, et al., Japanese Patent Application No. 98246347; Hasegawa, et al., Japanese Patent Application No. 84166283; and Yokoi, et al., Japanese Patent Application No. 85152468.

The entire contents of each of these references are herein expressly incorporated by reference, along with the foreign counterparts of the cited patents and patent applications; and all of the fredericamycin A compounds along with their methods of synthesis and selection discussed in the aforementioned references are intended to be part of this invention unless specifically stated otherwise.

Examples of fredericamycin A compounds follow. The fredericamycin A compounds are described below as several classes of compounds.

1st Class of Fredericamycin A Compounds (Described in Yokoi, et al., U.S. Pat. No. 4,584,377)

A fredericamycin A derivative of Formula I

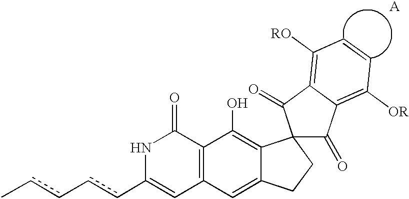

wherein R is a hydrogen atom or a C-acyl group, A denotes

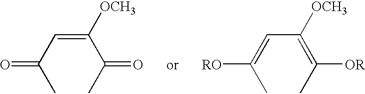

and the dotted lines in the formula indicate optional double bonds, with the proviso that when A is

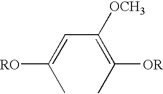

or when the optional double bonds are present in the formula, group R is a group other than a hydrogen atom.

2nd Class of Fredericamycin A Compounds (Described in Kelly, et al., U.S. Pat. No. 5,166,208)

A fredericamycin A derivative of Formula II

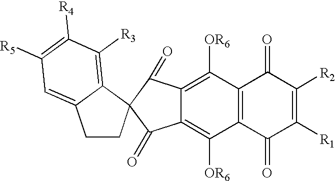

wherein

R₁ and R₂ are each independently selected from the group consisting of hydrogen, halo, hydroxy, arylthio having from 6 to 10 carbon atoms, alkylthio having from 1 to 8 carbon atoms, alkylthio having from 1 to 8 carbon atoms independently substituted at available positions by one or more hydroxy, halo, nitro, cyano, alkoxy having from 1 to 8 carbon atoms, amino, alkylamino having from 1 to 8 carbon atoms, C₁₋₈-alkoxycarbonylamino, guanidino, ureido, C₁₋₈-alkylureylene, alkanoylamino, C₁₋₈-alkoxycarboxyl, alkenyl having 2 to 6 carbons atoms, alkynyl having 2 to 6 carbon atoms, cycloalkyl having 3 to 7 ring members, cycloalkenyl having 5 to 7 ring members and a group of the formula —S—S—R′ wherein R′ is selected from the group consisting of alkyl having from 1 to 8 carbon atoms, cycloalkyl having from 3 to 7 ring members, alkanoylamino, aryl having from 6 to 10 carbon atoms, and aryl having from 6 to 10 carbon atoms substituted by alkyl having from 1 to 8 carbons atom, and a group of the Formula —N(R₇)R₈ wherein R₇ and R₈ are each independently selected from the group consisting of hydrogen, hydroxy, alkyl having from 1 to 8 carbon atoms, alkenyl having from 2 to 6 carbon atoms, alkynyl having from 2 to 6 carbon atoms, alkoxy having from 1 to 8 carbon atoms, C₁₋₈-alkoxycarbonyl, alkanoyl, cycloalkyl having 3 to 7 ring members, aryl having from 6 to 10 carbon atoms, aryl having from 6 to 10 carbon atoms substituted by alkyl having from 1 to 8 carbon atom, C₆₋₁₀-arylcarbonyl, amidino, and diakylaminocarbonyl having 3 to 12 carbon atoms;

R₃ is selected from the group consisting of hydrogen, hydroxy, alkyl having from 1 to 8 carbon atoms, and alkoxy having from 1 to 8 carbon atoms;

R₄ and R₅ together form a ring selected from the following Formulas IIA and IIB

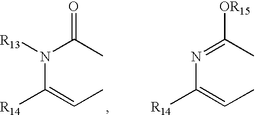

wherein R₁₃ is selected from the group consisting of hydrogen and alkyl having from 1 to 8 carbon atoms; R₁₄ is selected from the group consisting of alkyl having from 1 to 8 carbon atoms, alkenyl having from 2 to 8 carbon atoms, alkanoyl, and alkynyl having from 2 to 8 carbon atoms; R₁₅ is selected from the group consisting of hydrogen, alkyl having from 1 to 8 carbon atoms, and alkanoyl;

R₆ is selected from the group consisting of hydrogen, alkanoyl, C₆₋₁₀-aryl carbonyl, and a pharmaceutically acceptable cation; and pharmaceutically acceptable salts thereof.

3rd Class of Fredericamycin A Compounds (Described in Clive, et al. 1996. Tetrahedron 52:6085-6116)

A fredericamycin A derivative of Formula III

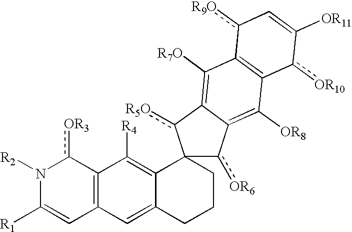

wherein the dotted lines indicate optional double bonds;

R₁ is alkyl having from 1 to 8 carbon atoms, alkenyl having from 2 to 8 carbon atoms, alkanoyl, or alkynyl having from 2 to 8 carbon atoms R₂ is hydrogen or alkyl having from 1 to 8 carbon atoms;

R₃, R₅, R₆, R₉, and R₁₀ are independently hydrogen, alkyl having from 1 to 8 carbon atoms, alkanoyl, or nothing; and

R₄, R₇, R₈, R₁₁ are independently hydrogen, alkyl having from 1 to 8 carbon atoms, or alkanoyl.

An example of a fredericamycin A derivative of Formula III (class 3) is Formula IV.

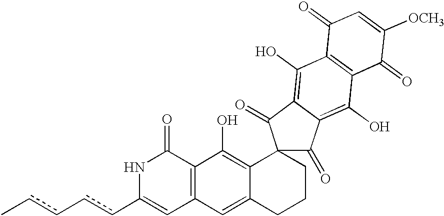

wherein the dotted lines indicate optional double bonds

An example of a fredericamycin A derivative of class 3 is Formula V

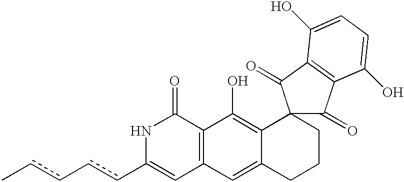

wherein the dotted lines indicate optional double bonds.

4th Class of Fredericamycin A Compounds (Purpuromycin Related Compounds)

A fredericamycin A derivative of Formula VI

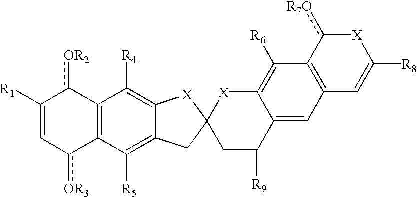

wherein the dotted lines indicate optional double bonds;

X is N, O, S, or C;

R₁, R₄, R₅, R₆, R₈, and R₉ are independently hydrogen, alkyl, hydroxyl, alkoxy, alkanoyl, alkoxycarbonyl, alkylcarbonyl, alkylcarbonyloxy, alkoxycarbonyloxy; and

R₂, R₃, and R₇ are independently hydrogen, alkyl, alkanoyl, or nothing.

An example of a fredericamycin A derivative of Formula VI (class 4) is Formula VII (purpuromycin).

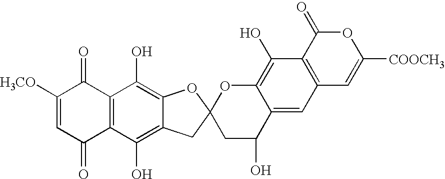

An example of a fredericamycin A derivative of class 4 is Formula VIII (heliquinomycin):

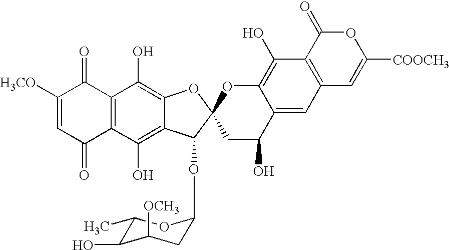

5th Class of Fredericamycin A Compounds

A fredericamycin A derivative of Formula IX

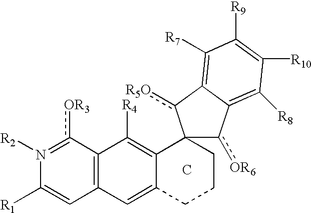

wherein the dotted lines around C indicate that C may be a 5 or 6 membered ring;

wherein the dotted lines not around C indicate optional double bonds;

R₁ is alkyl, alkenyl, alkanoyl, alknyl;

R₂ is hydrogen or alkyl;

R₉ and R₁₀ are both hydrogen or together form a ring having the structure

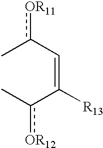

R₃, R₅, R₆, R₁₁, and R₁₂ are independently hydrogen, alkyl, alkanoyl, or nothing; and

R₄, R₇, R₈, R₁₃ are independently hydrogen, alkyl, hydroxyl, alkoxy, alkanoyl, alkoxycarbonyl, alkylcarbonyl, alkylcarbonyloxy, alkoxycarbonyloxy.

An example of a fredericamycin A derivative of Formula VIII is Formula X (fredericamycin A)

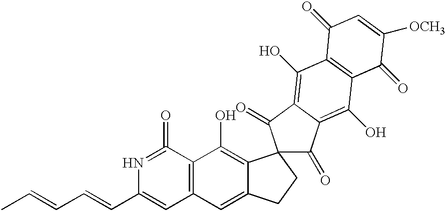

6th Class of Fredericamycin A Compounds

A fredericamycin A derivative of Formula XI

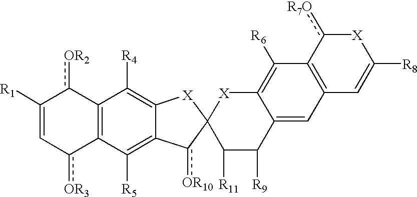

wherein the dotted lines indicate optional double bonds;

X is N, O, S, or C;

R₁, R₄, R₅, R₆, R₈, R₉, and R₁₁ are independently hydrogen, alkyl, hydroxyl, alkoxy, alkanoyl, alkoxycarbonyl, alkylcarbonyl, alkylcarbonyloxy, or alkoxycarbonyloxy, or R₉ and R₁₁ taken together form an epoxide ring; and

R₂, R₃, R₇, and R₁₀ are independently hydrogen, alkyl, alkanoyl, or nothing.

An example of a fredericamycin A derivative of Formula VI (class 4) is Formula VII (purpuromycin).

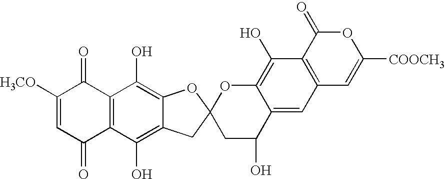

An example of a fredericamycin A derivative of class 6 is Formula VIII (heliquinomycin):

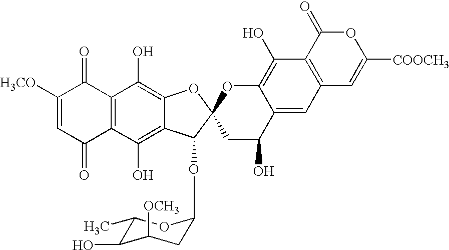

Other examples of fredericamycin A derivatives of class 6 include, but are not limited to, compounds of the formulae:

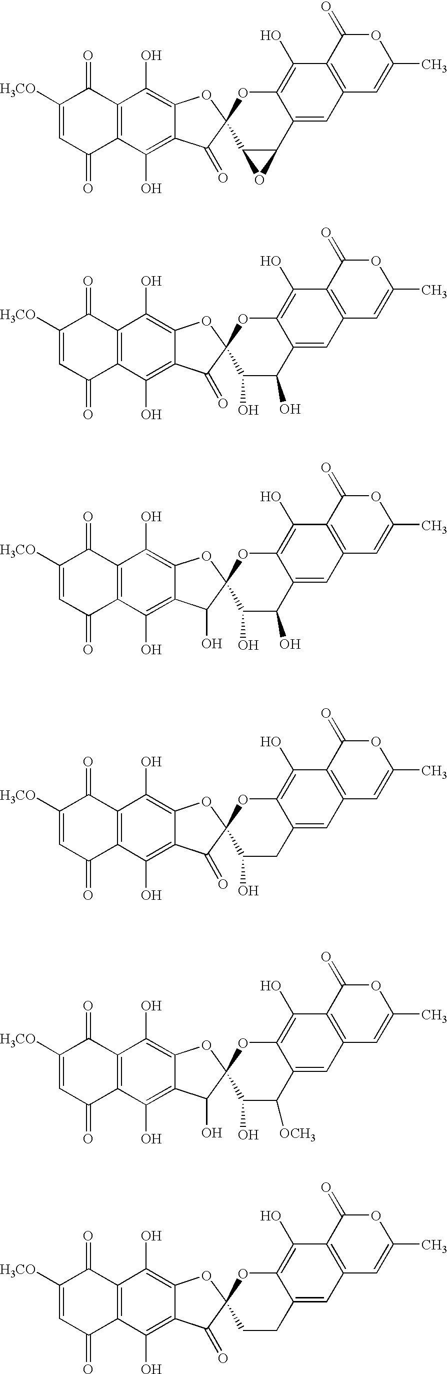

Treatment of Diseases or Disorders

The fredericamycin A compounds of the present invention be used to treat, inhibit, and/or prevent undesirable cell growth, neoplasia, and/or cancer in any subject but particularly in humans. The fredericamycin A compounds of the present invention be used to inhibit Pin1 activity in a subject. The fredericamycin A compounds of the present invention be used to inhibit cyclin D1 expression in a subject

Treatment of Neoplasms and Abnormal Cell Growth

The language “hyperplastic inhibitory agent” is intended to include agents that inhibit the growth of proliferating cells or tissue wherein the growth of such cells or tissues is undesirable. For example, the inhibition can be of the growth of malignant cells such as in neoplasms or benign cells such as in tissues where the growth is inappropriate. Examples of the types of agents which can be used include chemotherapeutic agents, radiation therapy treatments and associated radioactive compounds and methods, and immunotoxins.

The language “chemotherapeutic agent” is intended to include chemical reagents which inhibit the growth of proliferating cells or tissues wherein the growth of such cells or tissues is undesirable. Chemotherapeutic agents are well known in the art (see e.g., Gilman A. G., et al., The Pharmacological Basis of Therapeutics, 8th Ed., Sec 12:1202-1263 (1990)), and are typically used to treat neoplastic diseases. The chemotherapeutic agents generally employed in chemotherapy treatments are listed below in Table 1. Other similar examples of chemotherapeutic agents include: bleomycin, docetaxel (Taxotere), doxorubicin, edatrexate, etoposide, finasteride (Proscar), flutamide (Eulexin), gemcitabine (Gemzar), goserelin acetate (Zoladex), granisetron (Kytril), irinotecan (Campto/Camptosar), ondansetron (Zofran), paclitaxel (Taxol), pegaspargase (Oncaspar), pilocarpine hydrochloride (Salagen), porfimer sodium (Photofrin), interleukin-2 (Proleukin), rituximab (Rituxan), topotecan (Hycamtin), trastuzumab (Herceptin), tretinoin (Retin-A), Triapine, vincristine, and vinorelbine tartrate (Navelbine).

TABLE 1

| | | NONPROPRIETARY NAMES |
|---|---|---|
| CLASS | TYPE OF AGENT | (OTHER NAMES) |
| Alkylating | Nitrogen Mustards | Mechlorethamine (HN₂) |
| | | Cyclophosphamide |
| | | Ifosfamide |
| | | Melphalan (L-sarcolysin) |
| | | Chlorambucil |
| | Ethylenimines | Hexamethylmelamine |
| | And Methylmelamines | Thiotepa |
| | Alkyl Sulfonates | Busulfan |
| | Nitrosoureas | Carmustine (BCNU) |
| | | Lomustine (CCNU) |
| | | Semustine (methyl-CCNU) |
| | | Streptozocin (streptozotocin) |
| | Triazenes | Decarbazine (DTIC; |
| | | dimethyltriazenoimi- |
| | | dazolecarboxamide) |
| | Alkylator | cis-diamminedichloroplatinum |
| | | II (CDDP) |
| Antimeta- | Folic Acid Analogs | Methotrexate (amethopterin) |
| bolites | Pyrimidine | Fluorouracil (‘5-fluorouracil; 5- |
| | Analogs | FU); |
| | | Floxuridine (fluorode- |
| | | oxyuridine); |
| | | FUdr |
| | | Cytarabine (cyosine arabinoside) |
| | Purine Analogs | Mercaptopuine (6- |
| | and Related | mercaptopurine; 6-MP) |
| | Inhibitors | Thioguanine (6-thioguanine; TG) |
| | | Pentostatin (2'- |
| | | deoxycoformycin) |
| Natural | Vinca Alkaloids | Vinblastin (VLB) |
| Products | | Vincristine |
| | Topoisomerase | Etoposide |
| | Inhibitors | Teniposide |
| | Camptothecin | |
| | Topotecan | |
| | | 9-amino-campotothecin CPT-11 |
| | Antibiotics | Dactinomycin (actinomycin D) |
| | | Adriamycin |
| | | Daunorubicin (daunomycin; |
| | | rubindomycin) |
| | | Doxorubicin |
| | | Bleomycin |
| | | Plicamycin (mithramycin) |
| | | Mitomycin (mitomycin C) |
| | | Taxol |
| | | Taxotere |
| | Enzymes | L-Asparaginase |
| | Biological Response | Interfon alfa |
| | Modifiers | interleukin 2 |
| Miscella- | Platinum Coordination | cis-diamminedichloroplatinum II |
| neous | Complexes | (CDDP) |
| Agents | | Carboplatin |
| | Anthracendione | Mitoxantrone |
| | Substituted Urea | Hydroxyurea |
| | Methyl Hydraxzine | Procarbazine |
| | Derivative | (N-methylhydrazine, |
| | | (MIH) |
| | Adrenocortical | Mitotane (o,p' - DDD) |
| | Suppressant | Aminoglutethimide |
| Hormones | Adrenocorticosteroids | Prednisone |
| and | Progestins | Hydroxyprotesterone caproate |
| Antagon- | | Medroxyprogesterone acetate |
| ists | | Megestrol acetate |
| | Estrogens | Diethylstilbestrol |
| | | Ethinyl estradiol |
| | Antiestrogen | Tamoxifen |
| | Androgens | Testosterone propionate |
| | | Fluoxymesterone |
| | Antiandrogen | Flutamide |
| | Gonadotropin-releasing | Leuprolide |
| | Hormone analog | |

The language “radiation therapy” is intended to include the application of a genetically and somatically safe level of x-rays, both localized and non-localized, to a subject to inhibit, reduce, or prevent symptoms or conditions associated with undesirable cell growth. The term x-rays is intended to include clinically acceptable radioactive elements and isotopes thereof, as well as the radioactive emissions therefrom. Examples of the types of emissions include alpha rays, beta rays including hard betas, high energy electrons, and gamma rays. Radiation therapy is well known in the art (see e.g., Fishbach, F., Laboratory Diagnostic Tests, 3rd Ed., Ch. 10: 581-644 (1988)), and is typically used to treat neoplastic diseases.

The term “immunotoxins” includes immunotherapeutic agents which employ cytotoxic T cells and/or antibodies, e.g., monoclonal, polyclonal, phage antibodies, or fragments thereof, which are utilized in the selective destruction of undesirable rapidly proliferating cells. For example, immunotoxins can include antibody-toxin conjugates (e.g., Ab-ricin and Ab-diptheria toxin), antibody-radiolabels (e.g., Ab-I¹³⁵) and antibody activation of the complement at the tumor cell. The use of immunotoxins to inhibit, reduce, or prevent symptoms or conditions associated with neoplastic diseases are well known in the art (see e.g., Harlow, E. and Lane, D., Antibodies, (1988)).

Pin1-Associated States and Other Conditions

“Pin1-associated state” includes a disorder or a state (e.g., a disease state) which is associated with abnormal cell growth, abnormal cell proliferation, or aberrant levels of Pin1 marker. Pin1-associated state includes states resulting from an elevation in the expression of cyclin D1 and/or Pin1. Pin1-associated state also includes states resulting from an elevation in the phosphorylation level of c-Jun, particularly phosphorylation of c-Jun on S^{63/73}-P and/or from an elevation in the level of c-Jun amino terminal kinases (JNKs) present in a cell. Pin1-associated states include neoplasia, cancer, undesirable cell growth, and/or tumor growth. Pin1-associated state includes states caused by DNA damage, an oncogenic protein (i.e. Ha-Ras), loss of or reduced expression of a tumor suppressor (i.e. Brca1), and/or growth factors.

Pin1 is an important regulator of cyclin D1 expression. Because of Pin1's role in regulating the expression of cyclin D1, many of the tumor causing effects of cyclin D1 can be regulated through Pin1. In particular, inhibitors of Pin1 can be used to treat, inhibit, and/or prevent undesirable cell growth, neoplasia, and/or cancer in any subject but particularly in humans.

Pin1 is essential for cell growth; depletion or mutations of Pin1 cause growth arrest, affect cell cycle checkpoints and induce premature mitotic entry, mitotic arrest and apoptosis in human tumor cells, yeast or Xenopus extracts. Lu, et al. 1996. Nature 380:544-547. Winkler, et al. 2000. Science 287:1644-1647. Hani, et al. 1999. J. Biol. Chem. 274:108-116. Pin1 is dramatically overexpressed in human cancer samples and the levels of Pin1 are correlated with the aggressiveness of tumors. Furthermore, inhibition of Pin1 by various approaches, including the Pin1 inhibitor, Pin 1 antisense polynucleotides, or genetic depletion, kills human and yeast dividing cells by inducing premature mitotic entry and apoptosis. Pin1 is overexpressed in colon cancer cell lines, human breast cancer cell lines, and 75% of breast cancer tissues. Further, the levels of Pin1 correlate with the nuclear grade of the breast tumors and their cyclin D1 expression. These results indicate that the Pin-1 subfamily of enzymes is a promising new diagnostic and therapeutic target for diseases characterized by uncontrolled cell proliferation, primarily malignancies.

Pin1 is a highly conserved protein that binds and regulates the function of a defined subset of proteins that have been phosphorylated by Pro-directed kinases. Yaffe, et al. 1997. Science 278:1957-1960. Shen, et al. 1998. Genes Dev. 12:706-720. Lu, et al. 1999. Science 283:1325-1328. Crenshaw, et al. 1998. Embo J 17:1315-1327. Lu, et al. 1999. Nature 399:784-788. Zhou, et al. 1999 Cell Mol. Life Sci. 56:788-806. Pin1 contains an NH₂-terminl WW domain and a COOH-terminal peptidyl-prolyl isomerase (PPIase) domain. The WW domain binds specific pS/T-P motifs and targets Pin1 to its phosphoprotein substrates, where the PPIase domain regulates their conformations and functions, presumably by isomerizing specific pS/T-P bonds. Pin1 may cause the overexpression of endogenous cyclin D1. Pin1 is believed to activate the expression of cyclin D1 by acting cooperatively with c-Jun to activate the cyclin D1 promoter. In order to activate cyclin D1 expression, c-Jun must be phosphorylated. Pin1 binds to c-Jun mainly via phosophorylated S^{63/73}-P motifs. Pin1 activates phosphorylated c-Jun to induce cyclin D1 expression by regulating the conformation of the phosphorylated S-P motifs in c-Jun.

The activity of c-Jun is also enhanced by phosphorylation induced by growth factors, oncogenic proteins, DNA damage or other stress conditions. Although different pathways may be involved, they eventually lead to activation of Pro-directed kinasess, JNKs, which phosphorylate c-Jun on S^{63/73}-P and enhance its transcriptional activity. Binetruy, et al. 1991. Nature 351:122-127. Smeal, et al. 1991. Nature 354:494-496. Derijard, et al. 1994. Cell. 76:1025-1037. Thus, phosphorylation of c-Jun on S^{63/73}-P is a key regulatory mechanism that converts inputs from various signaling pathways into changes in cyclin D1 gene expression.

Oncogenic and tumor suppressor pathways may also affect the activity of Pin1. Pathways activated by oncogenic Ras may contribute to up-regulation of Pin1. Wildtype Brca (a tumor suppressor) suppresses the expression of Pin1.

“Increased cyclin D1 expression” or “cyclin D1 overexpression” or “elevation in the expression of cyclin D1”includes cells having higher than normal levels of cyclin D1. Significant cyclin D1 overexpression includes both small and large increases in the levels of cyclin D1 compared with normal levels. Preferably, cyclin D1 overexpression is considered in the context of the phase of the cell cycle. In actively proliferating normal cells, cyclin D1 reaches a peak in mid G₁ phase, decreases during S-phase, and remains low throughout the rest of the cycle. By contrast, in transformed cells the level of cyclin D1 is more variable. Therefore, cyclin D1 overexpression includes the expression of cyclin D1 at levels that are abnormally high for the particular cell cycle phase of the cell. Cyclin D1 overexpression can manifest itself as tumor growth or cancer. One skilled in the art would recognize that studies have been done measuring the level cyclin D1 expression in normal cells and cells having a cancerous state.

Increased cyclin D1 expression has been found in a vast range of primary human tumors. Increased cyclin D1 expression has been detected in the form of gene amplification, increased cyclin D1 RNA expression, and increased cyclin D1 protein expression. Most clinical studies comparing cyclin D1 gene amplification with expression of cyclin D1 have found that more cases show over-expression of both RNA and protein than show amplification of the gene. The presence of increased cyclin D1 RNA and/or protein expression without gene amplification suggests that other cellular genes such as pRb may affect the expression cyclin D1. Human tumors found to have increased cyclin D1 expression include: parathyroid adenomas, mantle cell lymphomas, breast cancers, head and neck squamous cell carcinomas (i.e. squamous carcinomas in the oral cavity, nasopharynx, pharynx, hypopharynx, and larynx), esophageal cancers, hepatocellular carcinomas, colorectal cancers, genitourinary cancers, lung cancers (i.e. squamous cell carcinomas of the lung), skins cancers (i.e. squamous cell carcinomas, melanomas, and malignant fibrous histiocytomas), sarcomas, and central nervous system malignancies (i.e. astrocytomas and glioblastomas), gastric adenocarcinomas, pancreatic adenocarcinomas, squamous carcinomas of the gall bladder. Donnellan, et al. 1998. J. Clin. Pathol: Mol. Pathol. 51:1-7. The cyclin D1 gene is amplified in approximately 20% of mammary carcinomas and the protein is overexpressed in approximately 50% of mammary carcinomas. Barnes, et al. 1998. Breast Cancer Research and Treatment. 52:1-15. Cyclin D1 overexpression in mantle cell lymphoma is discussed in Espinet, et al. 1999. Cancer Genet Cytogenet. 111(1):92-8 and Stamatopoulous, et al. 1999. Br. J Haematol. 105(1):190-7. Cyclin D1 overexpression in breast cancer is discussed in Fredersdorf, et al. 1997. PNAS 94(12):6380-5. Cyclin D1 overexpression in head and neck cancers is discussed in Matthias, et al. 1999. Cancer Epidemiol. Biomarkers Prev. 8(9):815-23; Matthias, et al. 1998. Clin. Cancer Res. 4(10):2411-8; and Kyomoto, et al. 1997. Int. J. Cancer. 74(6):576-81. Cyclin D1 overexpression in laryngeal carcinoma is discussed in Bellacosa, et al. 1996. Clin. Cancer Res. 2(l):175-80. Cyclin D1 overexpression in multiple myeloma is discussed in Hoechtlen-Vollmar, et al. 2000. Br. J. Haematol. 109(1):30-8; Pruneri, et al. 2000. Am. J. Pathol. 156(5):1505-13; and Janssen, et al. 2000. Blood 95(8):2691-8. It is believed that in many tumors, cyclin D1 acts in co-operation with other oncogenes or tumor suppressor genes.

Cyclin D1 expression is regulated by many factors. Growth factors (i.e. CSF1, platelet-derived growth factor, insulin-like growth factor, steroid hormones, prolactin, and serum stimulation) promote the synthesis of cyclin D1 and removal of growth factors will lead to a drop in cyclin D1 levels and arrest the cell in G₁. Hosokawa, et al. 1996. J Lab. Clin. Med. 127:246-52. Hypophosphorylated pRb stimulates cyclin D1 transcription. Cyclin D1 activity is inhibited by transforming growth factor β-1, p53, and cyclin dependent kinase inhibitors (CKIs). High levels of CKIs bind to cdks and reduce the ability of cyclins to activate the cdks. There are 2 classes of CKIs: the Kip/Cip family including p21, p27, and p57 and the INK4 family including p15, p16, 18, and p19. The Kip/Cip family members are capable of binding to and inhibiting most cyclin-cdk complexes, whereas the INK4 family members seem to be specific inhibitors of cyclin D1-cdk complexes. Donnellan, et al. 1998. J. Clin. Pathol: Mol. Pathol. 51:1-7. pRb and E2F are activators of CKI p16. TGF-β, cAMP, contact inhibition, and serum deprivation increase the levels of p27. Barnes, et al. 1998. Breast Cancer Research and Treatment. 52:1-15.

Cyclin D1 is believed to act through the phosphorylation of pRB. pRB is hypophosphorylated throughout the G₁ phase, phosphorylated just before the S phase, and remains phosphorylated until late mitosis. Hypophosphorylated pRB arrests cells in G₁ by forming a complex with the E2F family of DNA binding proteins. E2F transcription factors transcribe genes associated with DNA replication (the S phase of the cell cycle).

Cyclin D1 can form a complex with either cdk4 or cdk6 to form activated cdk4 or cdk6. Activated cdk4 or cdk6 induces the phosphorylation of pRb changing pRb from its hypophosphorylated form in which it binds to and inactivates E2F transcription factors to phosphorylated pRb which no longer binds to and inactivates E2F transcription factors. In some mouse lymphoma cells overexpressing D cyclins, pRb is hyperphosphorylated compared with pRb in cells not overexpressing D cyclins. It appears that cyclin Dl is required to initiate the phosphorylation of pRb and that event drives the cell through the restriction point at which stage the cell is committed to divide.

“Neoplasia” or “neoplastic transformation” is the pathologic process that results in the formation and growth of a neoplasm, tissue mass, or tumor. Such process includes uncontrolled cell growth, including either benign or malignant tumors. Neoplasms include abnormal masses of tissue, the growth of which exceeds and is uncoordinated with that of the normal tissues and persists in the same excessive manner after cessation of the stimuli which evoked the change. Neoplasms may show a partial or complete lack of structural organization and functional coordination with the normal tissue, and usually form a distinct mass of tissue. One cause of neoplasia is dysregulation of the cell cycle machinary.

Neoplasms tend to grow and function somewhat independently of the homeostatic mechanisms which control normal tissue growth and function. However, some neoplasms remain under the control of the homeostatic mechanisms which control normal tissue growth and function. For example, some neoplasms are estrogen sensitive and can be arrested by anti-estrogen therapy. Neoplasms can range in size from less than 1 cm to over 6 inches in diameter. A neoplasm even 1 cm in diameter can cause biliary obstructions and jaundice if it arises in and obstructs the ampulla of Vater.

Neoplasms tend to morphologically and functionally resemble the tissue from which they originated. For example, neoplasms arising within the islet tissue of the pancreas resemble the islet tissue, contain secretory granules, and secrete insulin. Clinical features of a neoplasm may result from the function of the tissue from which it originated. For example, excessive amounts of insulin can be produced by islet cell neoplasms resulting in hypoglycemia which, in turn, results in headaches and dizziness. However, some neoplasms show little morphological or functional resemblance to the tissue from which they originated. Some neoplasms result in such non-specific systemic effects as cachexia, increased susceptibility to infection, and fever.

By assessing the histologic and others features of a neoplasm, it can be determined whether the neoplasm is benign or malignant. Invasion and metastasis (the spread of the neoplasm to distant sites) are definitive attributes of malignancy. Despite the fact that benign neoplasms may attain enormous size, they remain discrete and distinct from the adjacent non-neoplastic tissue. Benign tumors are generally well circumscribed and round, have a capsule, and have a grey or white color, and a uniform texture. By contrast, malignant tumor generally have fingerlike projections, irregular margins, are not circumscribed, and have a variable color and texture. Benign tumors grow by pushing on adjacent tissue as they grow. As the benign tumor enlarges it compresses adjacent tissue, sometimes causing atrophy. The junction between a benign tumor and surrounding tissue may be converted to a fibrous connective tissue capsule allowing for easy surgical remove of benign tumors. By contrast, malignant tumors are locally invasive and grow into the adjacent tissues usually giving rise to irregular margins that are not encapsulated making it necessary to remove a wide margin of normal tissue for the surgical removal of malignant tumors. Benign neoplasms tends to grow more slowly than malignant tumors. Benign neoplasms also tend to be less autonomous than malignant tumors. Benign neoplasms tend to closely histologically resemble the tissue from which they originated. More high differentiated cancers, cancers that resemble the tissue from which they originated, tend to have a better prognosis than poorly differentiated cancers. Malignant tumors are more likely than benign tumors to have an aberrant function (i.e. the secretion of abnormal or excessive quantities of hormones).

The histological features of cancer are summarized by the term “anaplasia.” Malignant neoplasms often contain numerous mitotic cells. These cells are typically abnormal. Such mitotic aberrations account for some of the karyotypic abnormalities found in most cancers. Bizarre multinucleated cells are also seen in some cancers, especially those which are highly anaplastic.

“Dyplasia” refers to a pre-malignant state in which a tissue demonstrates histologic and cytologic features intermediate between normal and anaplastic. Dysplasia is often reversible.

“Anaplasia” refers to the histological features of cancer. These features include derangement of the normal tissue architecture, the crowding of cells, lack of cellular orientation termed dyspolarity, cellular heterogeneity in size and shape termed “pleomorphism.” The cytologic features of anaplasia include an increased nuclear-cytoplasmic ratio (nuclear-cytoplasmic ratio can be over 50% for maligant cells), nuclear pleomorphism, clumping of the nuclear chromatin along the nuclear membrane, increased staining of the nuclear chromatin, simplified endoplasmic reticulum, increased free ribosomes, pleomorphism of mitochondria, decrease in size and number of organelles, enlarged and increased numbers of nucleoli, and sometimes the presence of intermediate filaments.

As used herein, the term “cancer” includes a malignancy characterized by deregulated or uncontrolled cell growth, for instance carcinomas, sarcomas, leukemias, and lymphomas. The term “cancer” includes primary malignant tumors (e.g., those whose cells have not migrated to sites in the subject's body other than the site of the original tumor) and secondary malignant tumors (e.g., those arising from metastasis, the migration of tumor cells to secondary sites that are different from the site of the original tumor).

The term “carcinoma” includes malignancies of epithelial or endocrine tissues, including respiratory system carcinomas, gastrointestinal system carcinomas, genitourinary system carcinomas, testicular carcinomas, breast carcinomas, prostate carcinomas, endocrine system carcinomas, melanomas, choriocarcinoma, and carcinomas of the cervix, lung, head and neck, colon, and ovary. The term “carcinoma” also includes carcinosarcomas, which include malignant tumors composed of carcinomatous and sarcomatous tissues. An “adenocarcinoma” refers to a carcinoma derived from glandular tissue or a tumor in which the tumor cells form recognizable glandular structures.

The term “sarcoma” includes malignant tumors of mesodermal connective tissue, e.g., tumors of bone, fat, and cartilage.

The terms “leukemia” and “lymphoma” include malignancies of the hematopoietic cells of the bone marrow. Leukemias tend to proliferate as single cells, whereas lymphomas tend to proliferate as solid tumor masses. Examples of leukemias include acute myeloid leukemia (AML), acute promyelocytic leukemia, chronic myelogenous leukemia, mixed-lineage leukemia, acute monoblastic leukemia, acute lymphoblastic leukemia, acute non-lymphoblastic leukemia, blastic mantle cell leukemia, myelodyplastic syndrome, T cell leukemia, B cell leukemia, and chronic lymphocytic leukemia. Examples of lymphomas include Hodgkin's disease, non-Hodgkin's lymphoma, B cell lymphoma, epitheliotropic lymphoma, composite lymphoma, anaplastic large cell lymphoma, gastric and non-gastric mucosa-associated lymphoid tissue lymphoma, lymphoproliferative disease, T cell lymphoma, Burkitt's lymphoma, mantle cell lymphoma, diffuse large cell lymphoma, lymphoplasmacytoid lymphoma, and multiple myeloma.

For example, the therapeutic methods of the present invention can be applied to cancerous cells of mesenchymal origin, such as those producing sarcomas (e.g., fibrosarcoma, myxosarcoma, liosarcoma, chondrosarcoma, osteogenic sarcoma or chordosarcoma, angiosarcoma, endotheliosardcoma, lympangiosarcoma, synoviosarcoma or mesothelisosarcoma); leukemias and lymphomas such as granulocytic leukemia, monocytic leukemia, lymphocytic leukemia, malignant lymphoma, plasmocytoma, reticulum cell sarcoma, or Hodgkin's disease; sarcomas such as leiomysarcoma or rhabdomysarcoma, tumors of epithelial origin such as squamous cell carcinoma, basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, chorioaencinoma, semonoma, or embryonal carcinoma; and tumors of the nervous system including gioma, menigoma, medulloblastoma, schwannoma or epidymoma. Additional cell types amenable to treatment according to the methods described herein include those giving rise to mammary carcinomas, gastrointestinal carcinoma, such as colonic carcinomas, bladder carcinoma, prostate carcinoma, and squamous cell carcinoma of the neck and head region. Examples of cancers amenable to treatment according to the methods described herein include vaginal, cervical, and breast cancers.

The language “inhibiting undesirable cell growth” is intended to include the inhibition of undesirable or inappropriate cell growth. The inhibition is intended to include inhibition of proliferation including rapid proliferation. For example, the cell growth can result in benign masses or the inhibition of cell growth resulting in malignant tumors. Examples of benign conditions which result from inappropriate cell growth or angiogenesis are diabetic retinopathy, retrolental fibrioplasia, neovascular glaucoma, psoriasis, angiofibromas, rheumatoid arthritis, hemangiomas, Karposi's sarcoma, and other conditions or dysfunctions characterized by dysregulated endothelial cell division.

“Inhibiting tumor growth” or “inhibiting neoplasia” is intended to include the prevention of the growth of a tumor in a subject or a reduction in the growth of a pre-existing tumor in a subject. The inhibition also can be the inhibition of the metastasis of a tumor from one site to another. In particular, the language “tumor” is intended to encompass both in vitro and in vivo tumors that form in any organ or body part of the subject. The tumors preferably are tumors sensitive to the fredericamycin A compounds of the present invention. Examples of the types of tumors intended to be encompassed by the present invention include those tumors associated with breast cancer, skin cancer, bone cancer, prostate cancer, liver cancer, lung cancer, brain cancer, cancer of the larynx, gallbladder, esophagus, pancreas, rectum, parathyroid, thyroid, adrenal, neural tissue, head and neck, colon, stomach, bronchi, kidneys. Specifically, the tumors whose growth rate is inhibited by the present invention include basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuromas, intestinal ganglloneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multiforma, leukemias, lymphomas (i.e. maglinant lymphomas, mantle cell lymphoma), malignant melanomas, multiple myeloma, epidermoid carcinomas, and other carcinomas and sarcomas.

Administration of Fredericamycin A

The term “subject” is intended to include living organisms, e.g., prokaryotes and eukaryotes. Examples of subjects include mammals, e.g., humans, dogs, cows, horses, pigs, sheep, goats, cats, mice, rabbits, rats, and transgenic non-human animals. Most preferably the subject is a human.

The language “effective amount” of the compound is that amount necessary or sufficient to treat or prevent a Pin1 associated state, e.g. prevent the various morphological and somatic symptoms of a Pin1 associated state. In an example, an effective amount of the fredericamycin A compound is the amount sufficient to inhibit undesirable cell growth in a subject. In another example, an effective amount of the fredericamycin A compound is the amount sufficient to reduce the size of a pre-existing benign cell mass or malignant tumor in a subject. The effective amount can vary depending on such factors as the size and weight of the subject, the type of illness, or the particular Pin1 binding compound. For example, the choice of the Pin1 binding compound can affect what constitutes an “effective amount”. One of ordinary skill in the art would be able to study the aforementioned factors and make the determination regarding the effective amount of the Pin1 binding compound without undue experimentation. In one possible assay, an effective amount of a fredericamycin A compound can be determined by assaying for the expression of cyclin D1 and determining the amount of the fredericamycin A compound sufficient to reduce the levels of cyclin D1 to that associated with a non-cancerous state.

The regimen of administration can affect what constitutes an effective amount. The Pin1 binding compound can be administered to the subject either prior to or after the onset of a Pin1 associated state. Further, several divided dosages, as well as staggered dosages, can be administered daily or sequentially, or the dose can be continuously infused, or can be a bolus injection. Further, the dosages of the Pin1 binding compound(s) can be proportionally increased or decreased as indicated by the exigencies of the therapeutic or prophylactic situation.

The term “treated,” “treating” or “treatment” includes the diminishment or alleviation of at least one symptom associated or caused by the state, disorder or disease being treated. For example, treatment can be diminishment of one or several symptoms of a disorder or complete eradication of a disorder.

The language “pharmaceutical composition” includes preparations suitable for administration to mammals, e.g., humans. When the compounds of the present invention are administered as pharmaceuticals to mammals, e.g., humans, they can be given per se or as a pharmaceutical composition containing, for example, 0.1 to 99.5% (more preferably, 0.5 to 90%) of active ingredient in combination with a pharmaceutically acceptable carrier.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual, rectal, vaginal and/or parenteral administration. The formulations may conveniently be presented in unit dosage form and may be prepared by any methods well known in the art of pharmacy. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert dilutents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The preparations of the present invention may be given orally, parenterally, topically, or rectally. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, intravenous and subcutaneous doses of the compounds of this invention for a patient, when used for the indicated analgesic effects, will range from about 0.0001 to about 100 mg per kilogram of body weight per day, more preferably from about 0.01 to about 50 mg per kg per day, and still more preferably from about 1.0 to about 100 mg per kg per day. An effective amount is that amount treats an Pin1 associated state.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical composition.

The invention is further illustrated by the following examples, which should not be construed as further limiting. The contents of all references, pending patent applications and published patents, cited throughout this application are hereby expressly incorporated by reference. The animal models used throughout the Examples are accepted animal models and the demonstration of efficacy in these animal models is predictive of efficacy in humans.

Tumor Inhibition Assays

Fredericamycin A compounds are potent antitumor agents. The anti-tumor activity of fredericamycin A against glioblastoma cells is comparable to 1,3-bis(2-chloroethyl)-1-nitrosourea (BCNU), one of the most potent clinical useful antitumor agents. Misra, et al. 1982. J. Am. Chem. Soc. 104: 4478-4479.

In vitro anti-tumor activity of fredericamycin A compounds can be assayed by measuring the ability of fredericamycin A compounds to kill tumor cells. First allow an appropriate cell line to grow for a 24 hour period. Examples of appropriate cells lines include: human lung (A549); resistant human lung with low topo II activity (A549-VP); murine melanoma (B16); human colon tumor (HCT116); human colon tumor with elevated p170 levels (HCTVM); human colon tumor with low topo II activity (HCTVP); P388 murine lymph leukemia cells; and human colon carcinoma cell line (Moser). After the cells are allowed to attach for 24 hours to a plate (i.e. a 96-well flat bottom plate), the cells are incubated for 72 hours with serially diluted concentrations of fredericamycin A compounds. From these data, the concentration of the compound at which 50% of the cells are killed (IC₅₀) is determined. Kelly, et al., U.S. Pat. No. 5,166,208 and Pandey, et. al. 1981. J Antibiot. 34(11):1389-401.

In vivo anti-tumor activity of fredericamycin A compounds can be assayed for by a reduction of tumor cells in mammals (i.e. mice) and a resulting increase in survival time compared to untreated tumor bearing marnmals. For example, CDF₁ mice are injected interperitoneally with a suspension of P388 murine lymph leukemia cells, Ehrlich carcinoma cells, B16 melanoma cells, or Meth-A fibrosarcoma cells. Some of the mice are treated intraperitoneally with a fredericamycin A compounds. Other mice are treated with saline. The in vivo activity of the compound is determined in terms of the % T/C which is the ratio of the mean survival time of the treated group to the mean survival time of the saline treated group times 100. Yokoi, et al, U.S. Pat. No. 4,584,377; Kelly, et al., U.S. Pat. No. 5,166,208; Wamick-Pickle, et al. 1981. J Antibiot. 34(11):1402-7; and Pandey, et. al. 1981. J Antibiot. 34(11):1389-401.

The in vivo anti-tumor activity of fredericamycin A compounds can also be assayed as inhibitors against an ovarian tumor growing in a human tumor cloning system. Tebbe, et al. 1971 J. Am. Chem. Soc. 93:3793-3795.

The invention is further illustrated by the following examples, which should not be construed as further limiting. The contents of all references, pending patent applications and published patents, cited throughout this application are hereby expressly incorporated by reference.

Exemplification of the Invention:

### EXAMPLE 1

### Inhibition of the PPlase Activity of Pin1 by Fredericamycin A

1. Materials and Methods

PPIase activity measurements were performed by the protease-coupled PPIase assay developed by Fischer et al. (1984). For hPin1 activity measurements, bovine trypsin (final concentration 0.21 mg/mL, Sigma) was used as an isomer-specific protease and Ac-Ala-Ala-Ser(P)-Pro-Arg-pNA (Jerini, Germany) as a substrate. PPIase activity of hFKBP12 (Sigma) and hCyp18 (Sigma) was determined with the peptide substrate Suc-Ala-Phe-Pro-Phe-pNA (Bachem) and the protease α-chymotrypsin (final concentration 0.41 mg/mL, Sigma). The test was performed by observing the released 4-nitroanilide at 390 nm with a Hewlett-Packard 8453 UV-vis spectrophotometer at 10° C. The total reaction volume was adjusted to 1.23 mL by mixing appropriate volumes of 35 mM HEPES (pH 7.8) with enzyme and effector solutions. Fredericamycin A (BioLeads, Germany) was freshly diluted from a 1 mg/mL stock solution in DMSO. If not otherwise indicated, fredericamycin A (0-6 μM) was pre-incubated with the enzyme for 5 min (10° C.). Prior to the start of reaction by addition of the respective protease, 2 μL of the peptide substrate stock solution (10 mg/mL in DMSO) were added. The amount of organic solvent was kept constant within each experiment (<0.1%). The pseudo-first-order rate constant k_{obs} for cis/trans isomerization in the presence of PPlase and the first-order rate constant k₀ of the unkatalyzed cis/trans isomerization were calculated using the Kinetics Software of Hewlett-Packard as well as SigmaPlot2000 for Windows 6.0 (SPSS). The Kᵢ value for inhibition of hPin1 PPIase activity by fredericamycin A at constant concentrations of substrate ([S₀]<<K_{M}) was calculated by fitting the data according to the equation for a competitive “tight-binding” inhibitor using SigmaPlot2000.

2. Results

2.1 Determination of Kᵢ Value

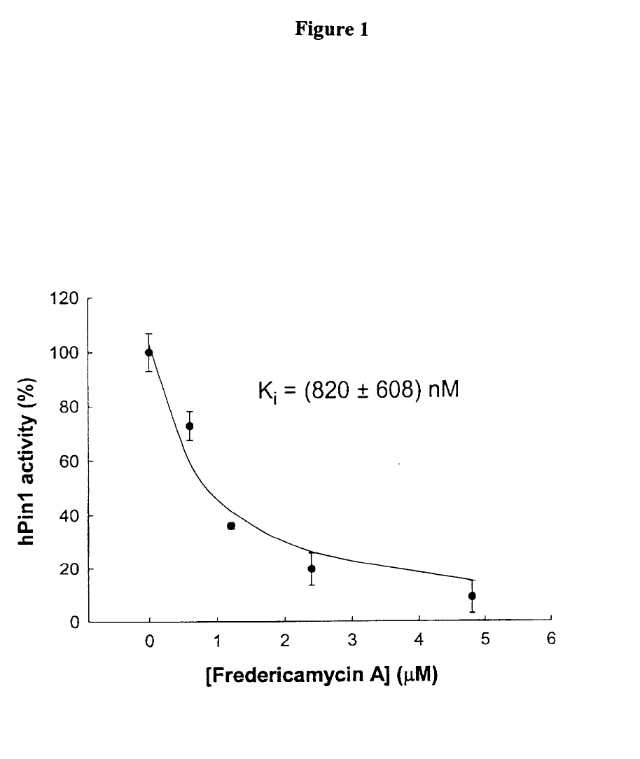

A Kᵢ value of (820±608) nM was determined for the inhibition of the PPIase activity of hPin1 by fredericamycin A (FIG. 1).

FIG. 1: Kᵢ value for hPin1 PPlase activity inhibition by fredericamycin A. PPIase activity measurements were performed as described in Materials and Methods. 6.0 nM of Pin1 were pre-incubated with 0-4.8 μM fredericamycin A in 35 mM HEPES (pH 7.8) at 110° C. for 5 min. Ac-Ala-Ala-Ser(P)-Pro-Arg-pNA (21.9 μM) was used as a substrate. Reactions were started by addition of trypsin.

2.2. Time Dependency of Inhibition of hPin1 PPIase Activity by Fredericamycin A

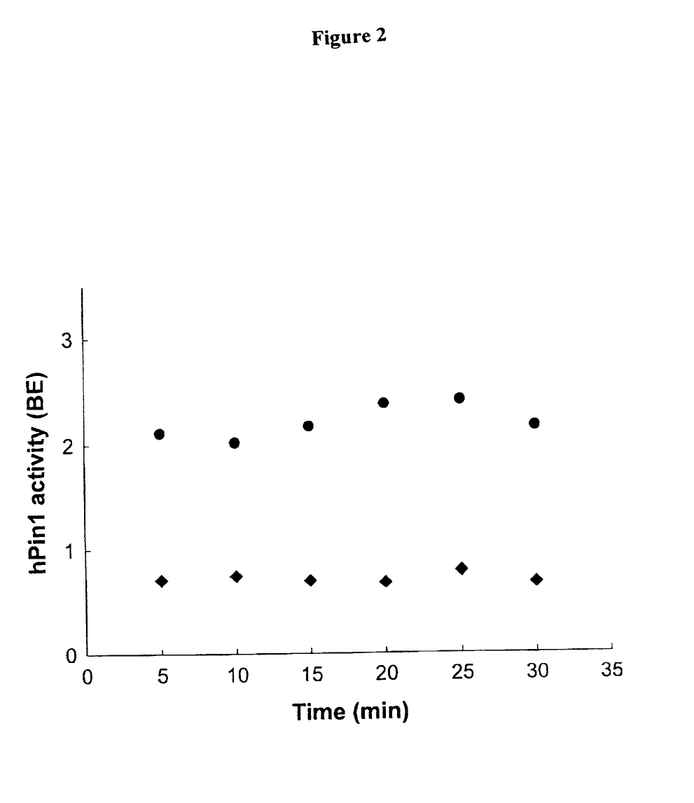

The time dependent changes of the PPIase activity of Pin1 (6.0 nM) upon addition of 0 and 1 μM of hPin1 were followed over a time interval of 30 min. As shown in FIG. 2, there was no progressive decrease of enzyme activity and thus no time dependency of inhibition within 30 min.

FIG. 2: Time dependency of Pin1 PPIase activity inhibition by fredericamycin A. PPIase activity measurements were performed as described in Materials and Methods. 6.0 nM hPin1 was incubated for 0, 5, 10, 15, 20, 25 and 30 min with 0 (●) and 1 μM (♦) fredericamycin, respectively.

2.3 Reversibility of the Inhibition of the PPlase Activity of hPin1 by Fredericamycin A

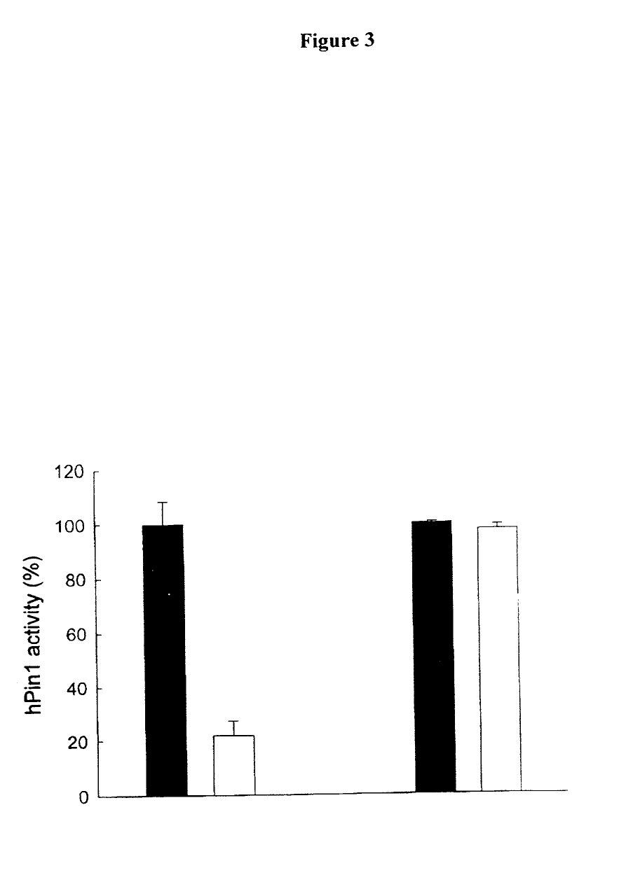

Reversibility of the interaction between hPin1 and fredericamycin A (FIG. 3) was performed by subjecting hPin1, (209 nM), which was inhibited up to 23% remaining activity by addition of 0.16 mM of fredericamycin, to micro-concentration through a semi-permeable membrane (microcon 10). After replacing the reaction buffer by 35 mM HEPES (pH 7.8) 3-times during the centrifugation, the remaining activity was assessed by the PPIase assay. Compared to the equivalently-treated inhibitor-free enzyme control, a hPin1 reactivation to 97.5% was observed, indicating reversibility of the binding of fredericamycin A to hPin1.

FIG. 3: Reversibility of the interaction between hPin1 and fredericamycin A. 209 nM of hPin1 were incubated with 0 (▪) and 0.16 (□) mM fredericamycin A and the remaining PPIase activity of hPin1 was measured before and after micro-separation through a semipermeable membrane using the protease-coupled PPlase assay according to Materials and Methods.

2.4 Specificity of the hPin1 PPIase Activity Inhibition by Fredericamycin A

Table 2 depicts the effect of fredericamycin A on the enzymatic activity of members of the three known families of PPIases: parvulins (hPin1), cyclophilins (hCyp18) and FKBPs (hFKBP12). In the protease-coupled PPIase assay, fredericamycin was identified as to inhibit all tested PPIases with an approximately 6- to 7-fold preference for the parvulin hPin1.

TABLE 2
Effect of fredericamycin A on the activity of hPin1, hFKBP12, hCyp18.

| PPIase | IC₅₀ (μM) |
|---|---|
| hPin1 | 0.89 ± 0.05 |
| hCyp18 | 5.1 ± 2.0 |
| hFKBP12 | 6.2 ± 1.7 |

### EXAMPLE 2

### Effect of Fredricamycin on DU-145 Prostate Tumor Bearing Scid Mice

The effects of Fredricamycin (FredA) on tumor growth in the scid mouse human prostate tumor model was studied. First, 44 scid mice were screened for immunoglobulin (Ig) production by ELISA. The mice were then inoculated with DU-145 prostate cancer cell line using a subcutaneous flank injection in sterile saline on Day 0.

On Day 16, 40 mice with established tumors (˜40 mm³) were selected and were divided into four groups of ten mice each. The first group received a dosage of the vehicle control (DMSO) on Days 16, 17, 18, 19, and 20. The second and third groups received dosages of 0.33 and 0.67 mg/kg of FredA, respectively, on Days 16, 17, 18, 19, and 20. The fourth group, a positive control, received Mitox at a dosage on 0.34 mg/kg also on Days 16, 17, 18, 19, and 20. Each of the dosages was administered via intraperitoneal injection.

On Days 1-56, the tumors in the mice were measured twice weekly and the volume of the tumors was estimated according to the formula: {(width)₂×length]/2. Mice were weighed before commencing the experiment and weekly thereafter to check for signs of toxicity.

None of the mice in the study receiving Mitox or the DMSO carrier alone died after 32 days. All of the mice receiving 0.67 mg/kg of Fred A died by about Day 17. Only 2 of the mice receiving 0.34 mg/kg of FredA lived until Day 32.

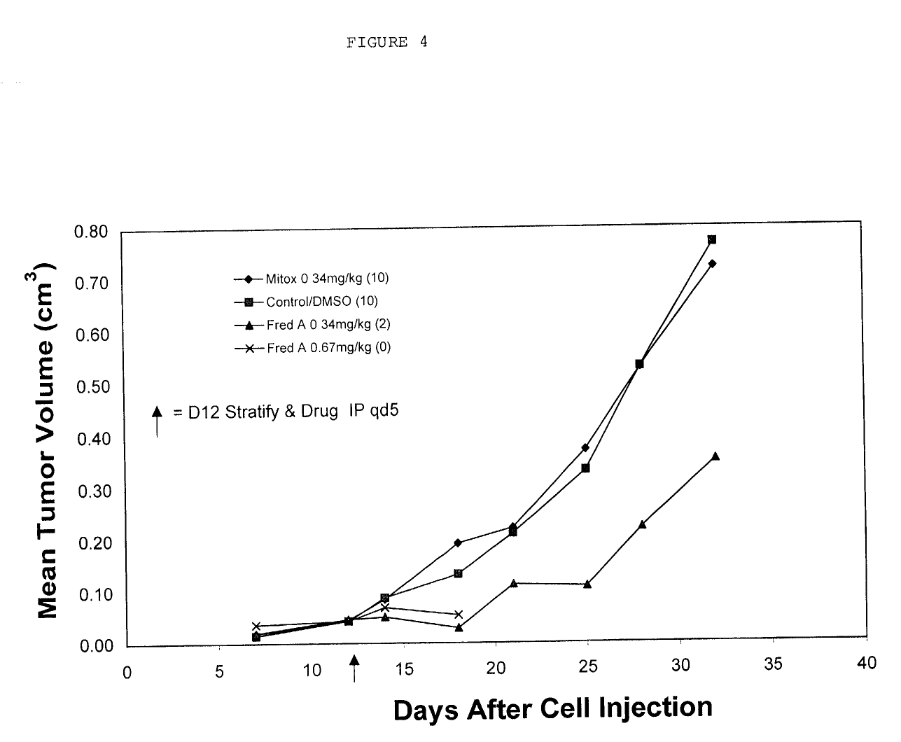

FIG. 4 is a line graph showing the mean tumor volume (cm³) over the trial period for each of the four groups. The figure shows that FredA was able to reduce the volume 50% as compared to the DMSO or Mitox controls. Table 3 summarizes the mean tumor volume data:

TABLE 3

| | Tumor Volume (cm³) | | | |
|---|---|---|---|---|
| | Control | Mitox | FredA | FredA |
| Day | DMSO | 0.34 mg/kg | 0.34 mg/kg | 0.67 mg/kg |
| 7 | 0.01 | 0.02 | 0.02 | 0.04 |
| 12 | 0.04 | 0.05 | 0.05 | 0.04 |
| 14 | 0.09 | 0.09 | 0.05 | 0.07 |
| 18 | 0.13 | 0.19 | 0.03 | 0.05 |
| 21 | 0.21 | 0.22 | 0.11 | — |
| 25 | 0.33 | 0.37 | 0.11 | — |
| 28 | 0.53 | 0.53 | 0.22 | — |
| 32 | 0.77 | 0.72 | 0.35 | — |

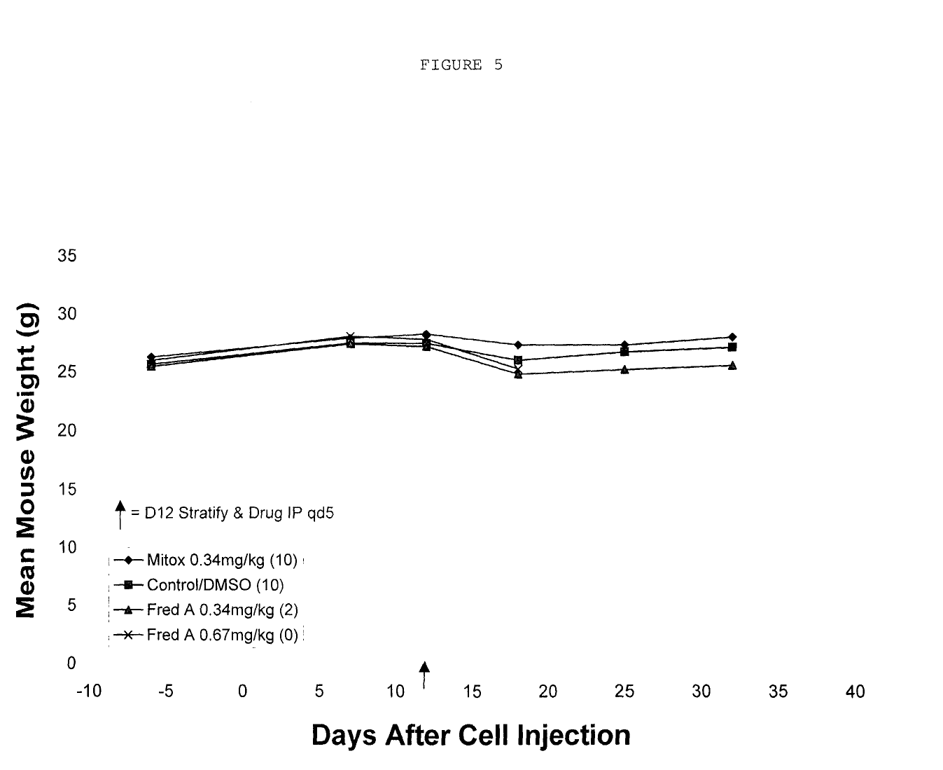

FIG. 5 is a line graph showing the mean mouse weight over the trial period for each of the four groups of mice. The figure shows that the mean weight of each of the four groups of mice remained generally consistent throughout the course of the experiment. Table 4 summarizes the mean tumor volume data:

TABLE 4

| | Mean Mouse Weight | | | |
|---|---|---|---|---|
| | Control | Mitox | FredA | FredA |
| Day | DMSO | 0.34 mg/kg | 0.34 mg/kg | 0.67 mg/kg |
| −6 | 25.73 | 26.36 | 25.53 | 26.08 |
| 7 | 27.56 | 27.98 | 27.47 | 28.14 |
| 12 | 27.52 | 28.32 | 27.24 | 27.86 |
| 18 | 26.05 | 27.39 | 24.86 | 25.30 |
| 25 | 26.77 | 27.38 | 25.25 | — |
| 32 | 27.17 | 28.04 | 25.60 | — |

Equivalents

Those skilled in the art will recognize, or be able to ascertain using no more than routine experimentation, many equivalents to the specific embodiments and methods described herein. Such equivalents are intended to be encompassed by the scope of the following claims.

1. A method for treating a Pin1-associated state in a subject comprising administering to a subject an effective amount of a fredericamycin A compound such that the Pin1-associated state is treated.

2. The method of claim 1, wherein the Pin1-associated state is a cyclin D1 elevated state.

3. The method of claim 1, wherein the Pin1-associated state is neoplastic transformation.

4. The method of claim 1, wherein the Pin1-associated state is cancer.

5. The method of claim 1, wherein the Pin1-associated state is tumor growth.

6. The method of claim 1, wherein the treating comprises inhibiting tumor growth.

7. The method of claim 1, wherein the treating comprises preventing the occurrence of tumor growth in the subject.

8. The method of claim 1, wherein the treating comprises reducing the growth of a pre-existing tumor in the subject.

9. The method of claim 1, wherein the Pin1-associated state is colon cancer.

10. The method of claim 1, wherein the Pin1-associated state is breast cancer.

11. The method of claim 1, wherein the Pin1-associated state is a sarcoma.

12. The method of claim 1, wherein the Pin1-associated state is a malignant lymphoma.

13. The method of claim 1, wherein the Pin1-associated state is esophageal cancer.

14. The method of claim 1, wherein the Pin1-associated state is caused by overexpression of Pin1.

15. The method of claim 1, wherein the Pin1-associated state is caused by DNA damage.

16. The method of claim 1, wherein the Pin1-associated state is caused by an oncogenic protein.

17. The method of claim 1, wherein the Pin1-associated state is caused by Ha-Ras.

18. The method of claim 1, wherein the fredericamycin A compound has Formula IX 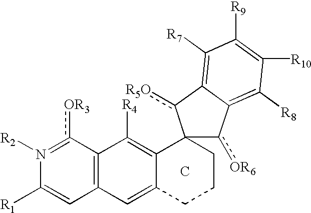
wherein the dotted lines around C indicate that C may be a 5 or 6 membered ring;
wherein the dotted lines not around C indicate optional double bonds;
R₁ is alkyl, alkenyl, alkanoyl, alknyl;
R₂ is hydrogen or alkyl;
R₉ and R₁₀ are both hydrogen or together form a ring having the structure 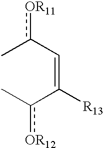
R₃, R₅, R₆, R₁₁, and R₁₂ are independently hydrogen, alkyl, alkanoyl, or nothing; and R₄, R₇, R₈, R₁₃ are independently hydrogen, alkyl, hydroxyl, alkoxy, alkanoyl, alkoxycarbonyl, alkylcarbonyl, alkylcarbonyloxy, alkoxycarbonyloxy, or pharmaceutically acceptable salts, ester, or prodrugs thereof.

19. The method of claim 1, wherein the fredericamycin A compound is fredericamycin A, or pharmaceutically acceptable salts, ester, or prodrugs thereof.

20. The method of claim 1, wherein the fredericamycin A compound has Formula III 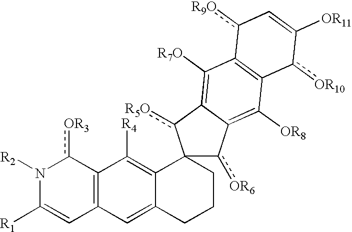
wherein the dotted lines indicate optional double bonds;
R₁ is alkyl having from 1 to 8 carbon atoms, alkenyl having from 2 to 8 carbon atoms, alkanoyl, or alkynyl having from 2 to 8 carbon atoms
R₂ is hydrogen or alkyl having from 1 to 8 carbon atoms;
R₃, R₅, R₆, R₉, and R₁₀ are independently hydrogen, alkyl having from 1 to 8 carbon atoms, alkanoyl, or nothing; and
R₄, R₇, R₈, R₁₁ are independently hydrogen, alkyl having from 1 to 8 carbon atoms, or alkanoyl, or pharmaceutically acceptable salts, ester, or prodrugs thereof.

21. The method of claim 1, wherein the fredericamycin A compound has Formula IV 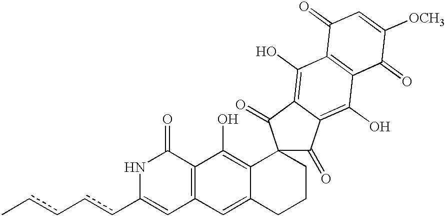
wherein the dotted lines indicate optional double bonds, or pharmaceutically acceptable salts, ester, or prodrugs thereof.

22. The method of claim 1, wherein the fredericamycin A compound has Formula VI 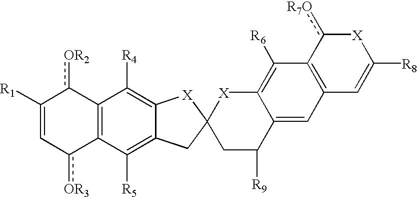
wherein the dotted lines indicate optional double bonds;
X is N, O, S, or C;
R₁, R₄, R₅, R₆, R₈, and R₉ are independently hydrogen, alkyl, hydroxyl, alkoxy, alkanoyl, alkoxycarbonyl, alkylcarbonyl, alkylcarbonyloxy, alkoxycarbonyloxy; and
R₂, R₃, and R₇ are independently hydrogen, alkyl, alkanoyl, or nothing, or pharmaceutically acceptable salts, ester, or prodrugs thereof.

23. The method of claim 1, wherein the fredericamycin A compound has Formula XI 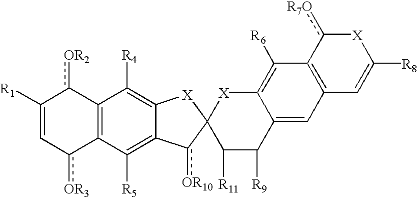
wherein the dotted lines indicate optional double bonds;
X is N, O, S, or C;
R₁, R₄, R₅, R₆, R₈, R₉, and R₁₁, are independently hydrogen, alkyl, hydroxyl, alkoxy, alkanoyl, alkoxycarbonyl, alkylcarbonyl, alkylcarbonyloxy, or alkoxycarbonyloxy, or R₉ and R₁₁ taken together form an epoxide ring; and
R₂, R₃, R₇, and R₁₀ are independently hydrogen, alkyl, alkanoyl, or nothing, or pharmaceutically acceptable salts, prodrugs, and esters thereof.

24. The method of claim 1, wherein the fredericamycin A compound has Formula VII 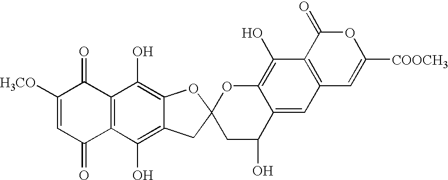
or pharmaceutically acceptable salts, ester, or prodrugs thereof.

25. The method of claims 1, wherein the fredericamycin A compound has Formula VIII: 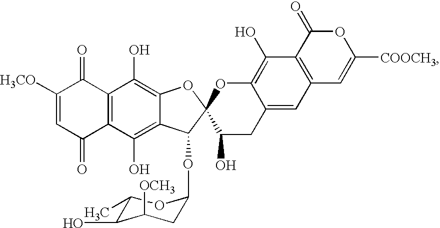
or pharmaceutically acceptable salts, ester, or prodrugs thereof.

26. The method of claim 1, wherein the fredericamycin A compound is a compound of the formulae: 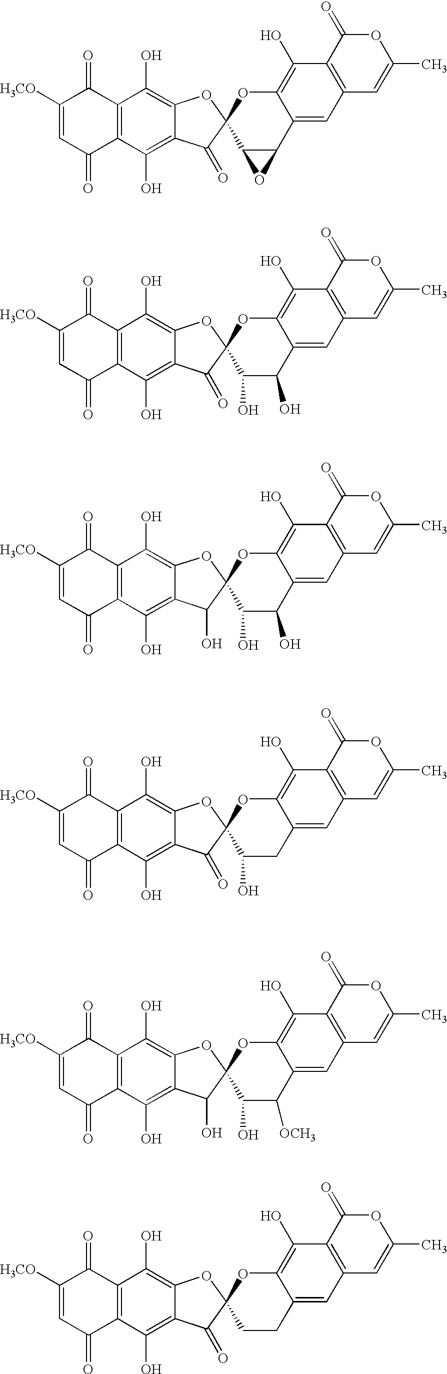
or pharmacuetically acceptable salts, prodrugs, and esters thereof.

27. The method of claim 1, wherein the fredericamycin A compound is a griseorhodin, or pharmacuetically acceptable salts, prodrugs, and esters thereof.

28. A method for treating cyclin D1 overexpression in a subject comprising administering to a subject an effective amount of a fredericamycin A compound such that cyclin D1 overexpression is treated.

29. The method of claim 28, wherein the cyclin D1 overexpression results in neoplastic transformation.

30. The method of claim 28, wherein the cyclin D1 overexpression results in tumor growth.

31. The method of claim 28, wherein the treating comprises inhibiting tumor growth.

32. The method of claim 28, wherein the treating comprises preventing the occurrence of tumor growth in the subject.

33. The method of claim 28, wherein the treating comprises reducing the growth of a pre-existing tumor in the subject.

34. The method of claim 28, wherein the cyclin D1 overexpression results in colon cancer.

35. The method of claim 28, wherein the cyclin D1 overexpression results in breast cancer.

36. The method of claim 28, wherein the cyclin D1 overexpression results in a sarcoma.

37. The method of claim 28, wherein the cyclin D1 overexpression results in a malignant lymphoma.

38. The method of claim 28, wherein cyclin D1 overexpression results in esophageal cancer.

39. The method of claim 28, wherein the cyclin D1 overexpression is caused by overexpression of Pin1.

40. The method of claim 28, wherein the cyclin D1 overexpression is caused by DNA damage.

41. The method of claim 28, wherein the cyclin D1 overexpression is caused by an oncogenic protein.

42. The method of claim 28, wherein cyclin Dl overexpression is caused by Ha-Ras.

43. The method of claim 28, wherein the fredericamycin A compound has Formula IX 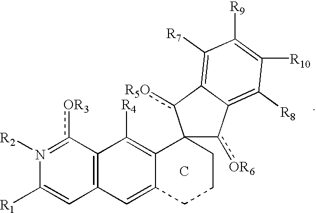
wherein the dotted lines around C indicate that C may be a 5 or 6 membered ring;
wherein the dotted lines not around C indicate optional double bonds;
R₁ is alkyl, alkenyl, alkanoyl, alknyl;
R₂ is hydrogen or alkyl;
R₉ and R₁₀ are both hydrogen or together form a ring having the structure 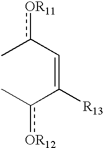
R₃, R₅, R₆, R₁₁, and R₁₂ are independently hydrogen, alkyl, alkanoyl, or nothing; and R₄, R₇, R₈, R₁₃ are independently hydrogen, alkyl, hydroxyl, alkoxy, alkanoyl, alkoxycarbonyl, alkylcarbonyl, alkylcarbonyloxy, alkoxycarbonyloxy, or pharmacuetically acceptable salts, prodrugs, and esters thereof.

44. The method of claim 28, wherein the fredericamycin A compound is fredericamycin A, or pharmacuetically acceptable salts, prodrugs, and esters thereof.

45. The method of claim 28, wherein the fredericamycin A compound has Formula III 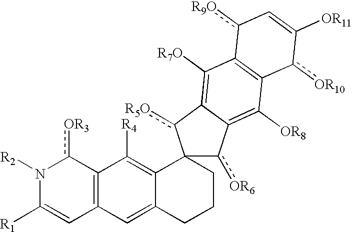
wherein the dotted lines indicate optional double bonds;
R₁ is alkyl having from 1 to 8 carbon atoms, alkenyl having from 2 to 8 carbon atoms, alkanoyl, or alkynyl having from 2 to 8 carbon atoms
R₂ is hydrogen or alkyl having from 1 to 8 carbon atoms;
R₃, R₅, R₆, R₉, and R₁₀ are independently hydrogen, alkyl having from 1 to 8 carbon atoms, alkanoyl, or nothing; and
R₄, R₇, R₈, R₁₁ are independently hydrogen, alkyl having from 1 to 8 carbon atoms, or alkanoyl, or pharmacuetically acceptable salts, prodrugs, and esters thereof.

46. The method of claim 28, wherein the fredericamycin A compound has Formula IV 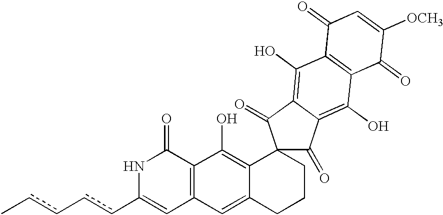
wherein the dotted lines indicate optional double bonds, or pharmacuetically acceptable salts, prod rugs, and esters thereof.

47. The method of claim 28, wherein the fredericamycin A compound has Formula VI 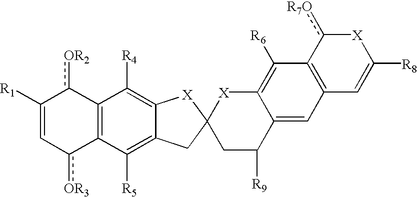
wherein the dotted lines indicate optional double bonds;
X is N, O, S, or C;
R₁, R₄, R₅, R₆, R₈, and R₉ are independently hydrogen, alkyl, hydroxyl, alkoxy, alkanoyl, alkoxycarbonyl, alkylcarbonyl, alkylcarbonyloxy, alkoxycarbonyloxy; and
R₂, R₃, and R₇ are independently hydrogen, alkyl, alkanoyl, or nothing, or pharmacuetically acceptable salts, prodrugs, and esters thereof.

48. The method of claim 28, wherein the fredericamycin A compound has Formula XI 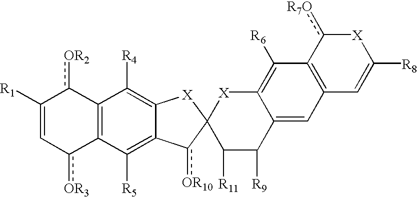
wherein the dotted lines indicate optional double bonds;
X is N, O, S, or C;
R₁, R₄, R₅, R₆, R₈, R₉, and R₁₁ are independently hydrogen, alkyl, hydroxyl, alkoxy, alkanoyl, alkoxycarbonyl, alkylcarbonyl, alkylcarbonyloxy, or alkoxycarbonyloxy, or R₉ and R₁₁ taken together form an epoxide ring; and
R₂, R₃, R₇, and R₁₀ are independently hydrogen, alkyl, alkanoyl, or nothing, or pharmaceutically acceptable salts, prodrugs, and esters thereof.

49. The method of claim 28, wherein the fredericamycin A compound has Formula VII 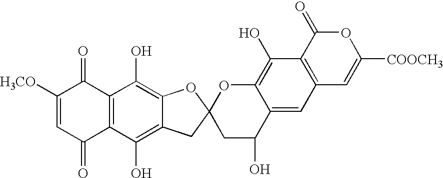
or pharmaceutically acceptable salts, prodrugs, and esters thereof.

50. The method of claim 28, wherein the fredericamycin A compound has Formula VIII 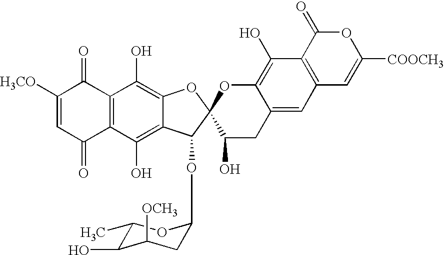
or pharmaceutically acceptable salts, prodrugs, and esters thereof.

51. The method of claim 28, wherein the fredericamycin A compound is a compound of the formulae: 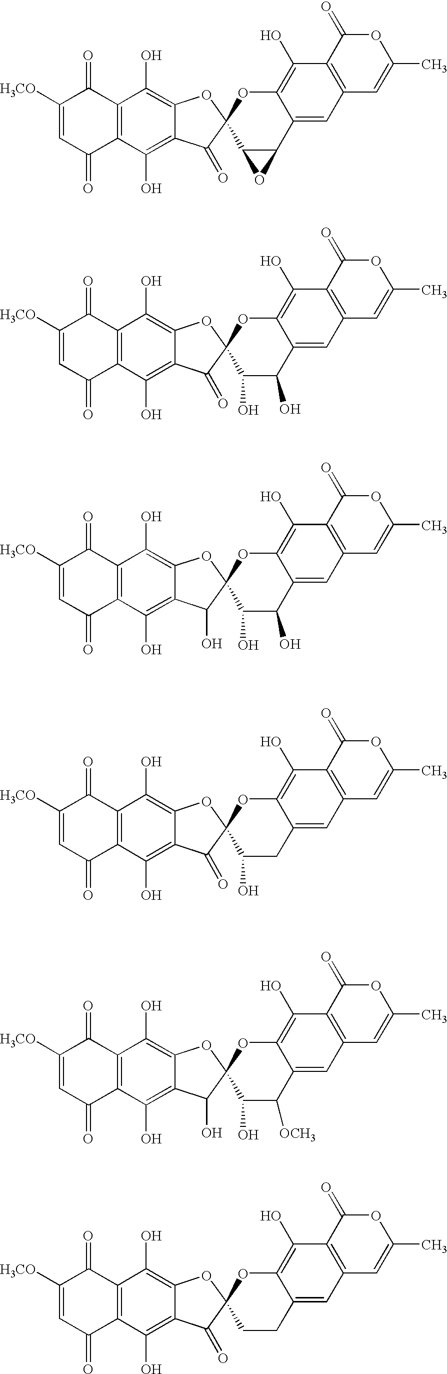
or pharmacuetically acceptable salts, prodrugs, and esters thereof.

52. The method of claim 28, wherein the fredericamycin A compound is a griseorhodin, or a pharmaceutically acceptable salt, prodrug or ester thereof.

53. A method for treating tumor growth in a subject comprising administering to a subject an effective amount of a fredericamycin A compound having Formula VI 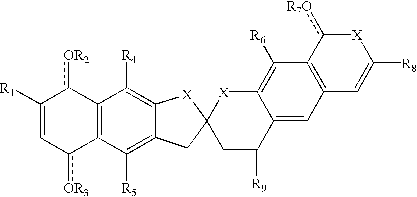
wherein the dotted lines indicate optional double bonds;
X is N, O, S, or C;
R₁, R₄, R₅, R₆, R₈, and R₉ are independently hydrogen, alkyl, hydroxyl, alkoxy, alkanoyl, alkoxycarbonyl, alkylcarbonyl, alkylcarbonyloxy, alkoxycarbonyloxy; and
R₂, R₃, and R₇ are independently hydrogen, alkyl, alkanoyl, or nothing, or pharmacuetically acceptable salts, prodrugs, and esters thereof; such that the tumor growth is treated.

54. The method of claim 53, wherein the treating comprises inhibiting tumor growth.

55. The method of claim 53, wherein the treating comprises preventing the occurrence of tumor growth in the subject.

56. The method of claim 53, wherein the treating comprises reducing the growth of a pre-existing tumor in the subject.

57. The method of claim 53, wherein the tumor growth is colon cancer.

58. The method of claim 53, wherein the tumor growth is breast cancer.

59. The method of claim 53, wherein the tumor growth is a sarcoma.

60. The method of claim 53, wherein the tumor growth is a malignant lymphoma.

61. The method of claim 53, wherein the tumor growth is esophageal cancer.

62. The method of claim 53, wherein the tumor growth is caused by overexpression of Pin1.

63. The method of claim 53, wherein the tumor growth is caused by DNA damage.

64. The method of claim 53, wherein the tumor growth is caused by an oncogenic protein.

65. The method of claim 53, wherein the tumor growth is caused by Ha-Ras.

66. The method of claim 53, wherein the tumor growth is caused by loss of Brca1 or a mutation of Brca1.

67. The method of claim 53, wherein the fredericamycin A compound has Formula VII:

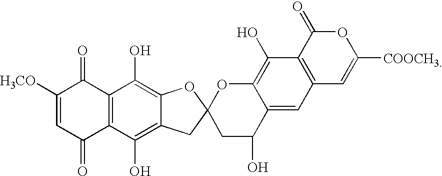

68. The method of claim 53, wherein the fredericamycin A compound is a griseorhodin, or a pharmaceutically acceptable salt, prodrug, or ester thereof.

69. A packaged Pin1-associated state treatment, comprising a fredericamycin A compound packaged with instructions for using an effective amount of the fredericamycin A compound to treat a Pin1-associated state.

70. A packaged cyclin D1 overexpression treatment, comprising a fredericamycin A compound packaged with instructions for using an effective amount of the fredericamycin A compound to treat cyclin D1 overexpression.

71. A packaged cancer treatment, comprising a fredericamycin A compound packaged with instructions for using an effective amount of the fredericamycin A compound to treat cancer.

72. A method for treating a Pin1-associated state in a subject comprising administering to a subject an effective amount of a combination of a fredericamycin A compound and a hyperplastic inhibitory agent such that the Pin1-associated state is treated.

73. The method of claim 72, wherein the fredericamycin A compound has Formula IX 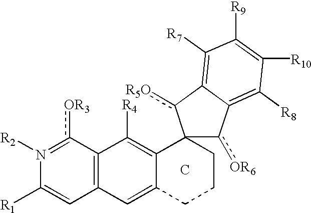
wherein the dotted lines around C indicate that C may be a 5 or 6 membered ring;
wherein the dotted lines not around C indicate optional double bonds;
R₁ is alkyl, alkenyl, alkanoyl, alknyl;
R₂ is hydrogen or alkyl;
R₉ and R₁₀ are both hydrogen or together form a ring having the structure 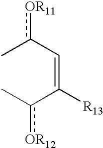
R₃, R₅, R₆, R₁₁, and R₁₂ are independently hydrogen, alkyl, alkanoyl, or nothing; and R₄, R₇, R₈, R₁₃ are independently hydrogen, alkyl, hydroxyl, alkoxy, alkanoyl, alkoxycarbonyl, alkylcarbonyl, alkylcarbonyloxy, alkoxycarbonyloxy, or pharmacuetically acceptable salts, prodrugs, and esters thereof.

74. The method of claim 72, wherein the fredericamycin A compound is fredericamycin A, or pharmacuetically acceptable salts, prodrugs, and esters thereof.

75. The method of claim 72, wherein the fredericamycin A compound has Formula III: 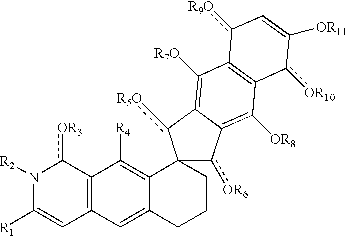
wherein the dotted lines indicate optional double bonds;
R₁ is alkyl having from 1 to 8 carbon atoms, alkenyl having from 2 to 8 carbon atoms, alkanoyl, or alkynyl having from 2 to 8 carbon atoms R₂ is hydrogen or alkyl having from 1 to 8 carbon atoms;
R₃, R₅, R₆, R₉, and R₁₀ are independently hydrogen, alkyl having from 1 to 8 carbon atoms, alkanoyl, or nothing; and
R₄, R₇, R₈, R₁₁ are independently hydrogen, alkyl having from 1 to 8 carbon atoms, or alkanoyl, or pharmacuetically acceptable salts, prodrugs, and esters thereof.

76. The method of claim 72, wherein the fredericamycin A compound has Formula IV: 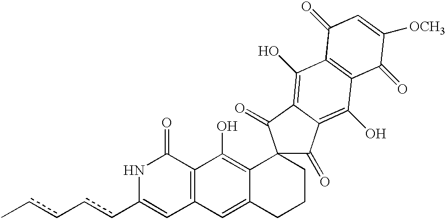
wherein the dotted lines indicate optional double bonds, or pharmacuetically acceptable salts, prodrugs, and esters thereof.

77. The method of claim 72, wherein the fredericamycin A compound has Formula VI: 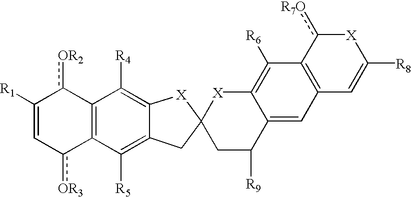
wherein the dotted lines indicate optional double bonds;
X is N, O, S, or C;
R₁, R₄, R₅, R₆, R₈, and R₉ are independently hydrogen, alkyl, hydroxyl, alkoxy, alkanoyl, alkoxycarbonyl, alkylcarbonyl, alkylcarbonyloxy, alkoxycarbonyloxy; and
R₂, R₃, and R₇ are independently hydrogen, alkyl, alkanoyl, or nothing, or pharmacuetically acceptable salts, prodrugs, and esters thereof.

78. The method of claim 72, wherein the fredericamycin A compound has Formula XI 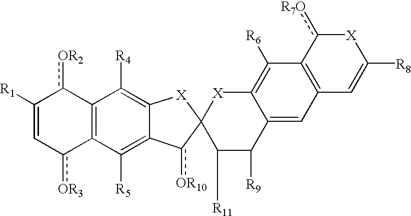
wherein the dotted lines indicate optional double bonds;
X is N, O, S, or C;
R₁, R₄, R₅, R₆, R₈, R₉, and R₁₁ are independently hydrogen, alkyl, hydroxyl, alkoxy, alkanoyl, alkoxycarbonyl, alkylcarbonyl, alkylcarbonyloxy, or alkoxycarbonyloxy, or R₉ and R₁₁ taken together form an epoxide ring; and
R₂, R₃, R₇, and R₁₀ are independently hydrogen, alkyl, alkanoyl, or nothing, or pharmaceutically acceptable salts, prodrugs, and esters thereof.

79. The method of claim 72, wherein the fredericamycin A compound has Formula VII 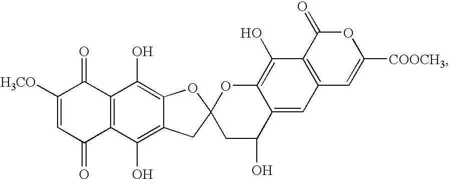
or pharmacuetically acceptable salts, prodrugs, and esters thereof.

80. The method of claim 72, wherein the fredericamycin A compound has Formula VIII: 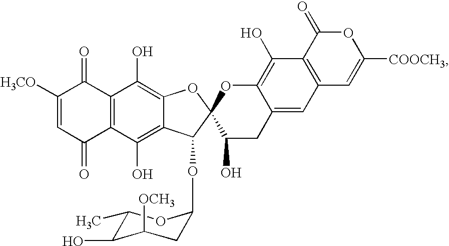
or pharmaceutically acceptable salts, prodrugs and esters thereof.

81. The method of claim 72, wherein the fredericamycin A compound is a compound of the formulae: 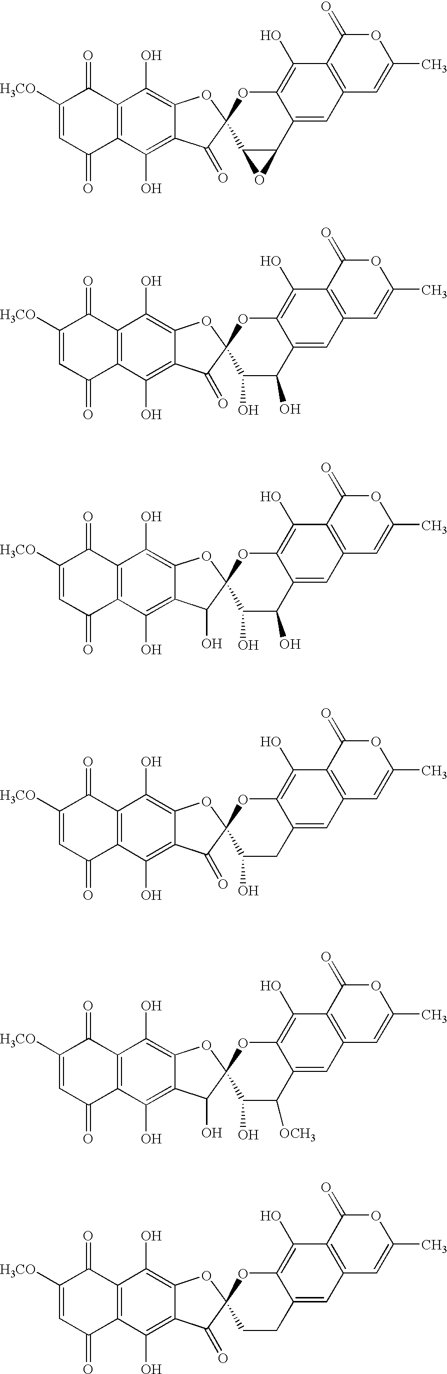
or pharmacuetically acceptable salts, prodrugs, and esters thereof.

82. The method of claim 72, wherein the fredericamycin A compound is a griseorhodin.

83. The method of claim 72, wherein the hyperplastic inhibitory agent is tamoxifen.

84. The method of claim 72, wherein the hyperplastic inhibitory agent is paclitaxel.

85. The method of claim 72, wherein the hyperplastic inhibitory agent is docetaxel.

86. The method of claim 72, wherein the hyperplastic inhibitory agent is interleukin-2.

87. The method of claim 72, wherein the hyperplastic inhibitory agent is rituximab.

88. The method of claim 72, wherein the hyperplastic inhibitory agent is tretinoin.

89. The method of claim 72, wherein the hyperplastic inhibitory agent methotrexate.

90. A method for treating cancer in a subject comprising administering to a subject an effective amount of a combination of a fredericamycin A compound and a hyperplastic inhibitory agent such that the cancer is treated.

91. A method for treating cyclin D1 overexpression in a subject comprising administering to a subject an effective amount of a combination of a fredericamycin A compound and a hyperplastic inhibitory agent such that the cyclin D1 overexpression is treated.
